(19) United States
(12) Patent Application Publication
(10) Pub. No.: US 2022/0226351 A1
(43) Pub. Date: Jul. 21, 2022

(54) Method of Controlling Progression of Hyperparathyroidism, And Compositions for Use Therein

(71) Applicant: EIRGEN PHARMA LTD., Waterford (IE)

(72) Inventor: Charles W. Bishop, Miami, FL (US)

(21) Appl. No.: 17/429,278

(22) PCT Filed: Feb. 6, 2020
(86) PCT No.: PCT/IB20/00089

§ 371 Date: Aug. 6, 2021

Related U.S. Application Data
(60) Provisional application No. 62/802,148, filed on Feb. 6, 2019.

(51) Int. Cl. A61K 31/592; A61K 31/593; A61P 5/18; A61P 3/02

(52) U.S. Cl. CPC A61K 31/592; A61P 3/02; A61P 5/18; A61K 31/593

(57) ABSTRACT

Methods and compositions for controlling hyperparathyroidism are disclosed.

### CROSS REFERENCE TO RELATED APPLICATIONS

The application is a U.S. National Phase of PCT/IB20/00089, filed Feb. 6, 2020, which claims the benefit under 35 U.S.C. § 119(e) of U.S. Provisional Application No. 62/802,148, filed Feb. 6, 2019, is hereby claimed and the entire contents thereof are incorporated herein by reference.

### BACKGROUND

### Field of the Disclosure

The disclosure relates generally to treatment of patients with increased serum intact parathyroid hormone, e.g. hyperparathyroidism. The disclosure also relates to treating SHPT, e.g. in Chronic Kidney Disease, and controlling progression of SHPT in Chronic Kidney Disease (CKD).

### Brief Description of Related Technology

SHPT is a disorder which develops primarily because of Vitamin D insufficiency (VDI) and deficiency. It is characterized by abnormally elevated blood levels of parathyroid hormone (PTH) and, in the absence of early detection and treatment, it becomes associated with parathyroid gland hyperplasia and a constellation of metabolic bone diseases. It is a common complication of CKD, with rising incidence as CKD progresses. SHPT can also develop in individuals with healthy kidneys, due to environmental, cultural or dietary factors which prevent adequate Vitamin D supply.

As to SHPT and its occurrence in CKD, there is a progressive loss of cells of the proximal nephrons, the primary site for the synthesis of the vitamin D hormones (collectively “1,25-dihydroxyvitamin D”) from 25-hydroxyvitamin D₃ and 25-hydroxyvitamin D₂. In addition, the loss of functioning nephrons leads to retention of excess phosphorus which reduces the activity of the renal 25-hydroxyvitamin D-1α-hydroxylase, the enzyme which catalyzes the reaction to produce the D hormones. These two events account for the low serum levels of 1,25-dihydroxyvitamin D commonly found in patients with moderate to severe CKD when Vitamin D supply is adequate.

CKD is characterized by overproduction of intact parathyroid hormone (iPTH) and hypertrophy of the parathyroid glands. It is associated with low serum total 25-hydroxyvitamin D, elevation of serum phosphorus and fibroblast growth factor 23 (FGF23), and decreased serum 1,25-dihydroxyvitamin D and calcium. Untreated, SHPT can lead to bone disease, increased fracture rates, vascular calcification, morbidity and mortality. Reduced serum levels of 1,25-dihydroxyvitamin D cause increased, and ultimately excessive, secretion of PTH by direct and indirect mechanisms. The resulting hyperparathyroidism leads to markedly increased bone turnover and its sequela of renal osteodystrophy, which may include a variety of other diseases, such as, osteitis fibrosa cystica, osteomalacia, osteoporosis, extraskeletal calcification and related disorders, e.g., bone pain, periarticular inflammation and Mockerberg's sclerosis. Reduced serum levels of 1,25-dihydroxyvitamin D also can cause muscle weakness and growth retardation with skeletal deformities (most often seen in pediatric patients).

Vitamin D compounds have traditionally been administered in immediate release formulations. Formulations for delivery of active vitamin D, analogs thereof, and prohormones thereof have been disclosed, including some extended release dosage forms. Some modified release dosage forms of vitamin D compounds have been described, e.g. in wax matrix form. One such formulation is marketed in the United States under the brand name RAYALDEE® (calcifediol), a product which is approved to treat SHPT in stage 3 and 4 CKD patients. The prescribing information for this drug provides that the sustained release formulation for RAYALDEE® is a wax based extended release formulation of 25-hydroxyvitamin D₃. See U.S. Patent Application Publication Nos. US 2009/311316 A1 (Dec. 17, 2009), US 2009/0176748 A1 (Jul. 9, 2009), US 2013/0137663 A1 (May 30, 2013), US 2014/0349979 A1 (Nov. 27, 2014), WO 2017/182237 A1 (Oct. 26, 2017), and U.S. Patent Application No. 62/725,940 (filed Aug. 31, 2018), the disclosures of which are incorporated herein by reference in their entireties.

Clinical practice guidelines target vitamin D sufficiency. Consensus, however, is lacking on the definition of vitamin D sufficiency in CKD. In 2003, the National Kidney Foundation (NKF) defined vitamin D sufficiency as serum total 25-hydroxyvitamin D concentrations of greater than 30 ng/mL and in 2011, the Endocrine Society defined it as concentrations between 30 and 100 ng/mL. The United States (US) Institute of Medicine (IOM) disagreed, stating in 2011 that “practically all persons are sufficient at serum 25-hydroxyvitamin D levels of at least 20 ng/mL.” The results described herein indicate that higher levels are required to control elevation of serum iPTH as CKD advances and to control progression of hyperparathyroidism.

### SUMMARY

One aspect of the disclosure provides a method for preventing, halting, or reversing SHPT progression in a subject, e.g. an adult human, defined as an increase in iPTH>10% from pre-treatment baseline, comprising effective administration of 25-hydroxyvitamin D to increase and maintain serum total 25-hydroxyvitamin D in the subject to a concentration greater than 50 ng/mL, optionally at least 50.8 ng/mL, optionally at least 51 ng/mL or at least 60 ng/mL and thereby prevent, halt, or reverse SHPT progression in the patient.

Another aspect of the disclosure provides a method of preventing, halting, or reversing SHPT progression in a population of patients, defined as an increase in plasma iPTH>10% from pre-treatment baseline, comprising effective administration of 25-hydroxyvitamin D to increase and maintain serum total 25-hydroxyvitamin D in the patients to a mean concentration greater than 50 ng/mL, optionally at least 50.8 ng/mL, optionally at least 51 ng/mL or at least 60 ng/mL, and thereby preventing, halting, or reversing SHPT progression in the patient population, wherein the fraction of subjects experiencing SHPT progression is less than 30%, 25%, 20%, 15%, 10%, or 9.7% or less, or less than 3%, or 2.8% or less.

A further aspect of the disclosure is a method of preventing, halting, or reversing SHPT progression in a patient, defined as an increase in plasma iPTH>10% from pre-treatment baseline, comprising: (a) increasing and maintaining serum total 25-hydroxyvitamin D in a patient; (b) decreasing serum iPTH in the patient, or (c) a combination thereof, to an extent better than that achieved with Vitamin D Analogs (VDA) or nutritional Vitamin D (NVD), hidroferol, or any combination thereof. Optionally, the method comprises: (a) increasing and maintaining serum total 25-hydroxyvitamin D in a patient; (b) decreasing serum iPTH in the patient, or (c) a combination thereof, to an extent which is at least 2-times that achieved with VDA, NVD, hidroferol, or any combination thereof. In various aspects, the serum total 25-hydroxyvitamin D is increased by more than 20 ng/mL compared to pre-treatment level. In various instances, the serum iPTH is decreased by at least 10 pg/mL, at least 20 pg/mL, or at least 30 pg/mL, compared to pre-treatment level. In various instances, the serum iPTH is decreased by more than 30% compared to pre-treatment level.

Additionally, an aspect of the disclosure is a method of preventing, halting, or reversing SHPT progression in a patient, defined as an increase in plasma iPTH>10% from pre-treatment baseline, comprising: (a) increasing and maintaining serum total 25-hydroxyvitamin D in a patient by greater than 20 ng/mL compared to pre-treatment level, (b) decreasing serum iPTH in the patient by at least 30% compared pre-treatment level, or (c) a combination thereof. In various instances of the presently disclosed methods of preventing, halting, or reversing of SHPT progression, the preventing, halting, or reversing of SHPT progression is achieved for 26 weeks or more.

Another aspect of the disclosure is a method of treating a disease, condition, or disorder associated with an increase in iPTH from baseline in a patient in need of treatment thereof, comprising effective administration of 25-hydroxyvitamin D to increase and maintain the patient's serum total 25-hydroxyvitamin D in a range of about 50 to about 300 ng/mL, optionally at least 50.8 ng/mL, optionally at least 51 ng/mL, optionally, about 60 ng/mL to about 300 ng/mL during chronic administration, and thereby treat the disease, condition, or disorder.

Another aspect of the disclosure is a method of mitigating SHPT progression in a patient in need of treatment thereof, comprising effective administration of 25-hydroxyvitamin D in a dosage amount in a range of 100 to 900 μg per week to gradually increase and then maintain the patient's serum total 25-hydroxyvitamin D level to a concentration in a range of about 50 to 300 ng/mL, optionally, at least 50.8 ng/mL, optionally, at least 51 ng/mL, optionally, about 60 ng/mL to about 300 ng/mL, and thereby mitigate progression of SHPT progression in the patient.

Another aspect of the disclosure is a method of treating a patient by (a) increasing and maintaining serum total 25-hydroxyvitamin D in a patient by more than 20 ng/mL, (b) decreasing serum iPTH in the patient by at least 30 pg/mL, or (c) any combination thereof, said method comprising administering to the patient an amount of 25-hydroxyvitamin D for a treatment time period of at least 6 months. In various instances of any one of the presently disclosed methods, serum calcium and phosphorus levels are not changed in the patient during the treatment time period.

Another aspect of the disclosure is a method of treating SHPT in a patient having CKD, comprising administering to the patient a dose of 25-hydroxyvitamin D selected based on the patient's weight and baseline serum 25-hydroxyvitamin D concentration, or based on the patient's weight and desired rise in serum 25-hydroxyvitamin D. In various aspects, the method comprises selecting the patient's dose to provide a post-treatment serum 25-hydroxyvitamin D concentration of at least 50 ng/ml, or at least 50.8 ng/ml, or at least 51 ng/ml, or at least 60 ng/ml. In various instances, the method comprises selecting the patient's dose to provide a steady state serum 25-hydroxyvitamin D concentration of at least 50 ng/ml, or at least 50.8 ng/ml, or at least 51 ng/ml, or at least 60 ng/ml. Optionally, the administration is by extended release, oral dosing. In various aspects, the dose is a daily dose. In various aspects, the dose (D) in mcg is a daily dose or equivalent to a daily dose selected as a function of the patient's body weight at initiation of therapy (W) in kilograms and desired rise in serum 25-hyroxyvitamin D (R) in ng/ml with scaling factor (F) according to the relationship D=(R×W)/F, wherein F is in a range of about 60 to about 80, or about 65 to about 75, or about 68 to about 72, or about 69 to about 71, or about 70. In various instances, the patient's body weight W is in a range of 50 kg to 180 kg. The patient has Stage 3 or Stage 4 CKD in some aspects. Also, in various aspects, the patient's dose is selected based on the patient's weight and baseline serum 25-hydroxyvitamin D concentration to provide post-treatment serum 25-hydroxyvitamin D concentration of at least 50 ng/ml, or at least 50.8 ng/ml, or at least 51 ng/ml, or at least 60 ng/ml. In exemplary instances, the method further provides a reduction in the patient's plasma iPTH concentration of at least 30% compared to pre-treatment baseline.

Another aspect of the disclosure is a pharmaceutical composition for use in a method described herein, for example, a pharmaceutical composition comprising 25-hydroxyvitamin D and a pharmaceutically acceptable excipient wherein the composition is administered to treat a disease or condition associated with an increase in iPTH from baseline and said administration increases and maintains serum levels of 25-hydroxyvitamin D to a range of about 50 to about 300 ng/mL, optionally at least 50.8 ng/mL, optionally at least 51 ng/mL, optionally, about 60 ng/mL to about 300 ng/mL, during chronic administration of said composition.

In various instances of any one of the methods of the disclosure, the method comprises increasing 25-hydroxyvitamin D to maintain serum total 25-hydroxyvitamin D level in the patient to a concentration in a range of greater than 50 ng/mL to about 300 ng/mL, optionally, about 60 ng/mL to about 300 ng/mL, or in a range of greater than 50 ng/mL to about 200 ng/mL, optionally, about 60 ng/mL to about 200 ng/mL, or in a range of greater than 50 ng/mL to about 100 ng/mL, optionally, about 60 ng/mL to about 100 ng/mL, optionally, over a period of at least 8 weeks, or at least 10 weeks, or at least 12 weeks, or at least 14 weeks. In various aspects, the administration of 25-hydroxyvitamin D comprises avoiding significant increase in the patient's corrected serum calcium level, serum phosphorous level, serum FGF23 level, or any combination thereof, compared to pre-treatment baseline.

In various aspects, the patient has a serum total 25-hydroxyvitamin D greater than or about 30 ng/mL at initiation of therapy. Optionally, the patient has serum total 25-hydroxyvitamin D greater than or about 40 ng/mL at initiation of therapy.

In various aspects of any one of the methods of the disclosure, the method comprises administering to the patient a dose of 25-hydroxyvitamin D which is selected based on the patient's body weight at initiation of therapy. In various instances, the dose is a daily dose or equivalent to a daily dose of about 0.1 mcg per kg of the patient's body weight at initiation of therapy to about 1 mcg per kg of the patient's body weight at initiation of therapy, optionally, a daily dose or equivalent to a daily dose of about 0.15 mcg per kg of the patient's body weight at initiation of therapy to about 0.85 mcg per kg of the patient's body weight at initiation of therapy. In exemplary aspects, the daily dose is about 0.4 mcg to about 0.8 mcg per kg of the patient's body weight at initiation of therapy. For example, the method comprises administering to the patient a starting dose of 60 mcg when the patient's body weight at initiation is greater than or equal to 140 kg.

In various aspects of any one of the methods of the disclosure, the SHPT progression (and lack thereof) is based on 26 or more weeks of treatment compared to patients who are (a) untreated; or (b) treated with active vitamin D therapy (optionally calcitriol, paricalcitol, or doxercalciferol); (c) treated with nutritional vitamin D (ergocalciferol and/or cholecalciferol) or (d) treated with hidroferol.

The subject can be one who is vitamin D insufficient at initiation of therapy, e.g. having serum total 25-hydroxyvitamin D less than 30 ng/mL. The amount of 25-hydroxyvitamin D administered can be effective to achieve a serum total 25-hydroxyvitamin D level in a patient, or the mean in the population, up to about 93 ng/mL, or up to 92.5 ng/mL, or up to about 90 ng/mL, or up to about 85 ng/mL, or up to about 80 ng/mL, or up to about 70 ng/mL, or up to about 69 ng/mL, or up to 68.9 ng/mL. The subject can include one having CKD Stage 3 to 5, or Stage 3 to 4, or Stage 5. The 25-hydroxyvitamin D administered can include, consist essentially of, or consist of 25-hydroxyvitamin D₃, a.k.a. calcifediol. The 25-hydroxyvitamin D can be administered by modified release, including by sustained release (a.k.a. extended release or prolonged release). The administration can be by any suitable route, e.g. oral, intravenous, or transdermal. The 25-hydroxyvitamin D can also be administered intravenously over an extended period of time, e.g. via gradual injection or infusion, for example over a period of at least 1 hour, optionally up to 5 hours. The administration route and/or schedule can be such that substantial induction of CYP24A1 is avoided, e.g. characterized by a VMR of 5 or less, or 4.8 or less. The dose of 25-hydroxyvitamin D can be provided on a daily or other episodic basis, e.g. 2 times per week, 3 times per week, or weekly, for example. As mentioned above, the dose amount is effective to increase and maintain serum total 25-hydroxyvitamin D in the subject to a concentration greater than 50 ng/mL, optionally at least 50.8 ng/mL, optionally at least 51 ng/mL, and can be, for example 30 μg daily, or 60 μg daily, or 90 μg daily. The 25-hydroxyvitamin D can be administered in a unit dose form comprising 30 μg to 1000 μg of 25-hydroxyvitamin D, or 30 μg to 600 μg of 25-hydroxyvitamin D, for example 30 μg, or 60 μg, or 90 μg, or 200 μg. In some embodiments, the method comprises administering 25-hydroxyvitamin D in a range of about 100 μg to about 900 μg per week or a range of about 300 μg to about 900 μg per week, e.g. 600 μg per week, optionally divided into two or three doses per week, e.g. three times per week at dialysis treatment.

For the methods, articles, and kits described herein, optional features, including but not limited to components, compositional ranges thereof, substituents, conditions, and steps, are contemplated to be selected from the various aspects, embodiments, and examples provided herein.

Further aspects and advantages will be apparent to those of ordinary skill in the art from a review of the following detailed description. While the methods are susceptible of embodiments in various forms, the description hereafter includes specific embodiments with the understanding that the disclosure is illustrative, and is not intended to limit the invention to the specific embodiments described herein.

### BRIEF DESCRIPTION OF THE DRAWINGS

For further facilitating the understanding of the present invention, three drawing figures are appended hereto.

FIG. 1 shows changes in serum 25-hydroxyvitamin D, 1,25-dihydroxyvitamin D, and plasma iPTH by treatment group and CKD Stage. Mean (SE) data from PP subjects at pre-treatment baseline (Week 0), Weeks 8-12 and Weeks 20-26 were analyzed by treatment group and CKD stage. Differences between active and corresponding placebo groups or between CKD stages were calculated by t-test. FIG. 1(A) shows serum total 25-hydroxyvitamin D (25-OH-D) FIG. 1(B) shows serum total 1,25-dihydroxyvitamin D (1,25(OH)2D) FIG. 1(C) shows plasma iPTH ¶ indicates significantly different from corresponding placebo group, p<0.05; indicates Significantly different from corresponding placebo group, p<0.01; ¶¶¶¶ indicates significantly different from corresponding placebo group, p<0.0001. In each of FIGS. 1(A) to 1(C), the bar graphs are presented in the order of Placebo-CKD₃, Placebo-CK4, ER-CKD 3, and ER-CKD4 from left to right in each grouping.

FIG. 2 shows an analysis of plasma iPTH by duration of treatment and post-treatment 25-hydroxyvitamin D quintile. Mean (SE) data from PP subjects were analyzed by duration of treatment [Baseline (Week 0), Week 12 (average of treatment weeks 8-12) and EAP (Efficacy Assessment Period, average of treatment weeks 20-26), FIG. 2(A)] within a given quintile (FIG. 2A and upper portion of FIG. 2B), and by post-treatment 25-hydroxyvitamin D quintile (n=71-72 in each) (lower portion of FIG. 2B). Differences from baseline (indicated in FIG. 2A) and from Quintile 1 at EAP (indicated in FIG. 2B) were calculated by ANOVA with subsequent Bonferroni's correction. ULN=upper limit of normal; * indicates significantly different from baseline, p<0.05; ** indicates significantly different from baseline, p<0.01; **** indicates significantly different from baseline, p<0.0001; ††† indicates significantly different from Quintile 1, p<0.0001.

FIG. 3 is an analysis of Plasma iPTH response rates by post-treatment 25-hydroxyvitamin D quintile. The proportion of per-protocol (PP) subjects achieving an iPTH response, defined as a mean decrease of ≥30% in plasma iPTH from pre-treatment baseline, was analyzed as a function of mean post-treatment serum total 25-hydroxyvitamin D quintile. ††\ indicates significantly different from Quintile 1, p<0.05.

FIG. 4 shows a patient distribution between study cohorts, in connection with Example 2 below.

FIGS. 5-8 show the relationship between patient weight and dose response in serum 25-hydroxyvitamin D levels following 12 weeks of treatment with 30 mcg daily ERC, in connection with Example 1 below.

### DETAILED DESCRIPTION

Described herein are materials and methods for preventing, mitigating, halting, or reversing progression of SHPT, treating diseases, conditions, or disorders associated with an increase in iPTH from baseline, and related compositions for such methods and uses.

The results described herein show that elevation of mean serum total 25-hydroxyvitamin D in CKD Stage 3 and 4 patients with extended release calcifediol (ERC) to levels as high as 92.5 ng/mL over a 26-week period had no adverse effects on mean serum calcium, phosphorus, FGF23, eGFR, VMR or the urine Ca:Cr ratio, and did not increase mean serum 1,25-dihydroxyvitamin D above the upper limit of normal (ULN, 62 pg/mL). Extension of these studies to 52 weeks of ERC treatment demonstrated no increased risks related to these parameters. A positive correlation was observed between serum total 25-hydroxyvitamin D and 1,25-dihydroxyvitamin D, but no correlation was observed between serum total 25-hydroxyvitamin D and serum calcium or phosphorus.

Mean levels of serum total 25-hydroxyvitamin D of at least 50.8 ng/mL, and achieving those levels as described herein, are associated with proportional increases in serum 1,25-hydroxyvitamin D, and decreases in plasma iPTH and serum bone turnover markers, attenuating SHPT progression defined as increase in EOT iPTH>10% from pre-treatment baseline, and not associated with adverse changes in mean serum calcium, phosphorus, FGF23, eGFR or the urine Ca:Cr ratio.

Increasing 25-hydroxyvitamin D exposures as described herein also not only attenuates the progressive rise in serum bone turnover markers, but actually reduced the levels of these markers, suggesting improved control of high turnover bone disease and a reduction in the risk of related adverse sequelae. Bone degradation and resulting fractures are a significant source of morbidity and mortality in CKD patients with SHPT. Even mildly elevated PTH has recently been demonstrated to produce significant changes in bone architecture and reduce BMD at the spine. Poor bone health has been strongly associated with vascular calcification and the associated high rates of cardiovascular morbidity and mortality in CKD fostering considerable interest in improving bone health and reducing healthcare costs by diagnosing and correcting bone disease in patients with kidney disease.

Another aspect of the methods herein is normalizing plasma iPTH, e.g. in Stage 3 or 4, or 5 CKD patients having SHPT, by raising serum total 25-hydroxyvitamin D in a patient to greater than 92.5 ng/mL by a method described herein, e.g. to a level of at least 95 ng/mL, or at least 100 ng/mL, or at least 125 ng/mL, or at least 150 ng/mL, at least 175 ng/mL, at least than 200 ng/mL, without disturbing calcium metabolism, or phosphorous metabolism, or a marker thereof, or any combination of the foregoing. The method can include repeat dosing to achieve a serum 25-D level in a range of about 120 ng/mL to about 200 ng/mL, or about 120 ng/mL to about 160 ng/mL, or about 150 ng/mL to about 200 ng/mL, for example.

The materials and methods are contemplated to include embodiments including any combination of one or more of the additional optional elements, features, and steps further described below, unless stated otherwise.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “comprising” indicates the potential inclusion of other agents, elements, steps, or features, in addition to those specified.

As used herein, “Vitamin D insufficiency and deficiency” is generally defined as having serum total 25-hydroxyvitamin D level below 30 ng/mL.

As used herein “hypercalcemia” refers to condition in a patient wherein the patient has corrected serum level of calcium above 10.2 mg/dL. Normal corrected serum level of calcium for a human is between about 8.6 to 10.2 mg/dL. As used herein the term “hypercalciuria” refers to a condition in a patient wherein the patient has urinary calcium excretion of greater than 275 mg in men and greater than 250 mg in women. In the alternative, hypercalciuria can be defined as daily urinary excretion of more than 4 mg calcium per kg body weight. As another alternative, hypercalciuria can be defined as 24-hour urinary calcium concentration greater than 200 mg calcium per liter urine.

As used herein the term “hyperphosphatemia” refers to a condition in a patient having serum phosphorous level above 4.6 mg/dL.

As used herein the term 25-hydroxyvitamin D refers generically to forms of 25-hydroxyvitamin D, including 25-hydroxyvitamin D₂, 25-hydroxyvitamin D₃ and 25-hydroxyvitamin D₄. In any method described herein, it is contemplated that use of 25-hydroxyvitamin D can include, consist of, or consist essentially of a combination of 25-hydroxyvitamin D₂ and 25-hydroxyvitamin D₃. In any method described herein, it is contemplated that use of 25-hydroxyvitamin D can include, consist of, or consist essentially of 25-hydroxyvitamin D₃.

As used herein, the term “serum total 25-hydroxyvitamin D” refers to the sum of 25-hydroxyvitamin D₂ and 25-hydroxyvitamin D₃ in serum.

As used herein the term 1,25-dihydroxyvitamin D refers generically to forms of 25-hydroxyvitamin D, including 1,25-dihydroxyvitamin D₂, and 1,25-dihydroxyvitamin D₃. The term “serum total 1,25-dihydroxyvitamin D” refers to the sum of 1,25-dihydroxyvitamin D₂ and 1,25-dihydroxyvitamin D₃ in serum.

In any method or use according to the present disclosure, effective administration of 25-hydroxyvitamin D can include administration in an amount of 30 to 150 μg on the average per day, for example. For example, daily doses can be 30 μg, 60 μg, 90 μg, or 120 μg. Individual doses can be in a range of 5 to 1,000 μg, for example.

The 25-hydroxyvitamin D can be dosed on any suitable schedule. For example, the dosing schedule can be daily, or less frequently, for example or every other day, or two times per week, or three times per week, or weekly, or biweekly. The effective administration can include administering 25-hydroxyvitamin D in a range of about 100 μg to about 900 μg per week or a range of about 300 μg to about 900 μg per week, optionally 600 μg per week. The weekly dose can be divided, for example into two or three doses per week. For example, the dose can be given three times per week at dialysis treatment.

The 25-hydroxyvitamin D can be dosed with food, or without food, or without regard to food. In one type of embodiment, the 25-hydroxyvitamin D is dosed without food, e.g. at bedtime, to reduce variances in 25-hydroxyvitamin D absorption due to food.

In any method or use according to the present disclosure, the method comprises administering to the patient a dose of 25-hydroxyvitamin D which is selected based on the patient's body weight at initiation of therapy, and further optionally based on the patient's serum 25-hydroxyvitamin D level at initiation of therapy and/or the desired rise in the patient's serum 25-hydroxyvitamin D as a result of therapy. In various aspects, the dose is a daily dose, or equivalent to a daily dose of about 0.1 mcg per kg of the patient's body weight at initiation of therapy to about 1 mcg per kg of the patient's body weight at initiation of therapy, optionally, about 0.15 mcg per kg of the patient's body weight at initiation of therapy to about 0.85 mcg per kg of the patient's body weight at initiation of therapy. In some aspects, the daily dose is about 0.4 mcg to about 0.8 mcg per kg of the patient's body weight at initiation of therapy, optionally, the method comprises administering to the patient a starting dose of 60 mcg when the patient's body weight at initiation is greater than or equal to 140 kg.

Another aspect of the disclosure is a method of treating SHPT in a patient having CKD, comprising administering to the patient a dose of 25-hydroxyvitamin D selected based on the patient's weight and baseline serum 25-hydroxyvitamin D concentration, or based on the patient's weight and desired rise in serum 25-hydroxyvitamin D. In various aspects, the method comprises selecting the patient's dose to provide a post-treatment serum 25-hydroxyvitamin D concentration of at least 50 ng/ml, or at least 50.8 ng/ml, or at least 51 ng/ml, or at least 60 ng/ml. In various instances, the method comprises selecting the patient's dose to provide a steady state serum 25-hydroxyvitamin D concentration of at least 50 ng/ml, or at least 50.8 ng/ml, or at least 51 ng/ml, or at least 60 ng/ml. Optionally, the administration is by extended release, oral dosing. In various aspects, the dose is a daily dose. In various aspects, the dose (D) in mcg is a daily dose or equivalent to a daily dose selected as a function of the patient's body weight at initiation of therapy (W) in kilograms and desired rise in serum 25-hyroxyvitamin D (R) in ng/ml with scaling factor (F) according to the relationship D=(R×W)/F, wherein F is in a range of about 60 to about 80, or about 65 to about 75, or about 68 to about 72, or about 69 to about 71, or about 70. In various instances, the patient's body weight W is in a range of 50 kg to 180 kg. The patient has Stage 3 or Stage 4 CKD in some aspects. Also, in various aspects, the patient's dose is selected based on the patient's weight and baseline serum 25-hydroxyvitamin D concentration to provide post-treatment serum 25-hydroxyvitamin D concentration of at least 50 ng/ml, or at least 50.8 ng/ml, or at least 51 ng/ml, or at least 60 ng/ml. In exemplary instances, the method further provides a reduction in the patient's plasma iPTH concentration of at least 30% compared to pre-treatment baseline.

In any method or use according to the present disclosure, effective administration of 25-hydroxyvitamin D can include administering 25-hydroxyvitamin D to increase serum total 25-hydroxyvitamin D to a level of greater than 50 ng/mL, optionally at least 50.8 ng/mL, optionally at least 51 ng/mL or at least 60 ng/mL. From a population perspective, the fraction of subjects experiencing SHPT progression can be less than 30%, 25%, 20%, 15%, 10%, or 9.7% or less, or less than 3%, or 2.8% or less, for example. The amount of 25-hydroxyvitamin D administered can be effective to achieve a serum total 25-hydroxyvitamin D level in a patient, or the mean in the population, up to up 300 ng/mL, or up to 200 ng/mL, or up to 150 ng/mL, or up to 120 ng/mL, or up to 100 ng/mL, or up to about 93 ng/mL, or up to 92.5 ng/mL, or up to about 90 ng/mL, or up to about 85 ng/mL, or up to about 80 ng/mL, or up to about 70 ng/mL, or up to about 69 ng/mL, or up to 68.9 ng/mL, and further without causing hypercalcemia, hyperphosphatemia, and/or hypercalciuria. For example, the method can include increasing 25-hydroxyvitamin D to maintain serum total 25-hydroxyvitamin D level in the patient to a concentration in a range of greater than 50 ng/mL to about 300 ng/mL, or greater than 50 ng/mL to about 200 ng/mL, or greater than 50 ng/mL to about 100 ng/mL, optionally, in a range of 60 ng/mL to about 300 ng/mL, or greater than 60 ng/mL to about 200 ng/mL, or greater than 60 ng/mL to about 100 ng/mL. The method can include 25-hydroxyvitamin D therapy to increase serum total 25-hydroxyvitamin D to such levels and/or by such amounts for at least 12 weeks, or at least 19 weeks, or at least 26 weeks following the start of 25-hydroxyvitamin D therapy, and can continue for any desired period of time for example, at least 39 weeks, or at least 52 weeks or longer. In various aspects, the prevention, halting, or reversing of SHPT progression is achieved for 26 weeks or more. The method can include 25-hydroxyvitamin D therapy to increase serum total 25-hydroxyvitamin D to such levels and/or by such amounts as a therapeutic target range, e.g. to maintain steady state serum total 25-hydroxyvitamin D levels with such ranges.

In various aspects, the SHPT progression is based on 26 weeks of treatment compared to patients who are (a) untreated; or (b) treated with active vitamin D therapy (optionally calcitriol, paricalcitol, or doxercalciferol); (c) treated with nutritional vitamin D (ergocalciferol and/or cholecalciferol) or (d) treated with hidroferol. For example, the comparison in SHPT progression after 26 weeks of treatment can be to patients receiving 1 mcg paricalcitol daily, or patients receiving 0.25 μg calcitriol per day, or patients receiving 0.5 μg calcitriol per day, or patients receiving 0.25 μg doxercalciferol per day, or patients receiving ergocalciferol (14,000 IU per day, or 35,000 IU per day, or 50,000 IU per day, or 105,000 IU per day), or patients receiving cholecalciferol (5,000 IU per day, or 7,000 IU per day, or 14,000 IU per day, or 28,000 IU per day, or 35,000 IU per day, or 50,000 IU per day). Also, a method of preventing, halting, or reversing SHPT progression in a patient, defined as an increase in plasma iPTH>10% from pre-treatment baseline, is provided, wherein said comprises: (a) increasing and maintaining serum total 25-hydroxyvitamin D in a patient; (b) decreasing serum iPTH in the patient, or (c) a combination thereof, to an extent better than that achieved with Vitamin D Analogs (VDA) or nutritional Vitamin D (NVD), hidroferol, or any combination thereof. Optionally, the method comprises: (a) increasing and maintaining serum total 25-hydroxyvitamin D in a patient; (b) decreasing serum iPTH in the patient, or (c) a combination thereof, to an extent which is at least 2-times that achieved with VDA, NVD, hidroferol, or any combination thereof. In various aspects, the serum total 25-hydroxyvitamin D is increased by more than 20 ng/mL compared to pre-treatment level. In various instances, the serum iPTH is decreased by at least 10 pg/mL, at least 20 pg/mL, or at least 30 pg/mL, compared to pre-treatment level. In various instances, the serum iPTH is decreased by more than 30% compared to pre-treatment level.

Additionally provided is a method of preventing, halting, or reversing SHPT progression in a patient, defined as an increase in plasma iPTH>10% from pre-treatment baseline, comprising: (a) increasing and maintaining serum total 25-hydroxyvitamin D in a patient by greater than 20 ng/mL compared to pre-treatment level, (b) decreasing serum iPTH in the patient by at least 30% compared pre-treatment level, or (c) a combination thereof. In various instances of the presently disclosed methods of preventing, halting, or reversing of SHPT progression, the preventing, halting, or reversing of SHPT progression is achieved for 26 weeks or more.

Further provided is a method of treating a patient by (a) increasing and maintaining serum total 25-hydroxyvitamin D in a patient by more than 20 ng/mL, (b) decreasing serum iPTH in the patient by at least 30 pg/mL, or (c) any combination thereof, said method comprising administering to the patient an amount of 25-hydroxyvitamin D for a treatment time period of at least 6 months.

Another aspect of the disclosure is a method of treating patients with SHPT and CKD (e.g. Stage 3 or Stage 4) with doses of 25-hydroxyvitamin D (e.g. 25-hydroxyvitamin D₃) that are selected based on the patient's weight to give a desired increase (rise) in the patient's serum 25-hydroxyvitamin D level. In addition or in the alternative is a method of treating patients with SHPT and CKD (e.g. Stage 3 or Stage 4) with doses of 25-hydroxyvitamin D (e.g. 25-hydroxyvitamin D₃) that are selected based on the patient's weight and baseline serum 25-hydroxyvitamin D concentration pre-treatment, e.g. to result in a desired serum 25-hydroxyvitamin D level post-treatment or at steady state.

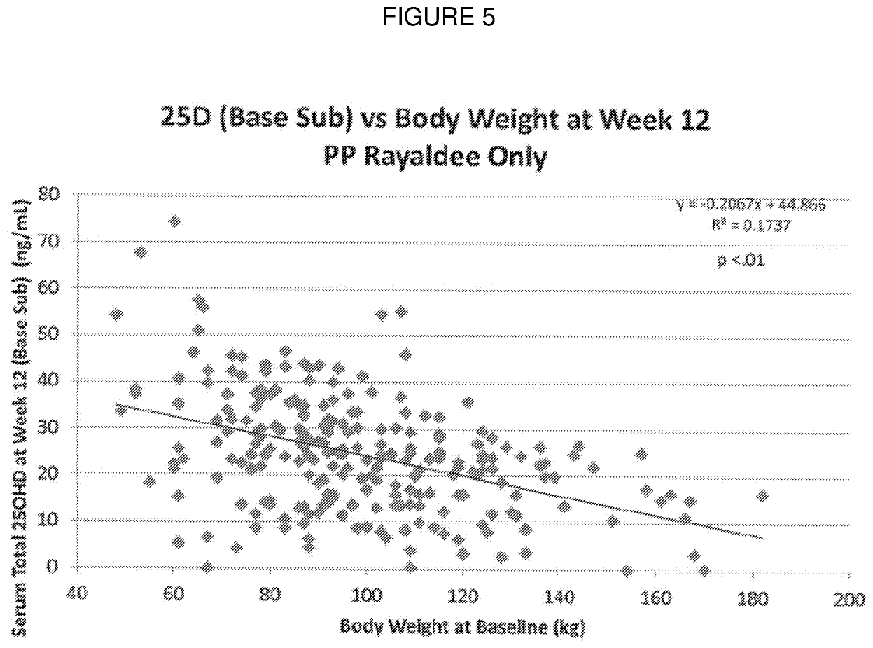

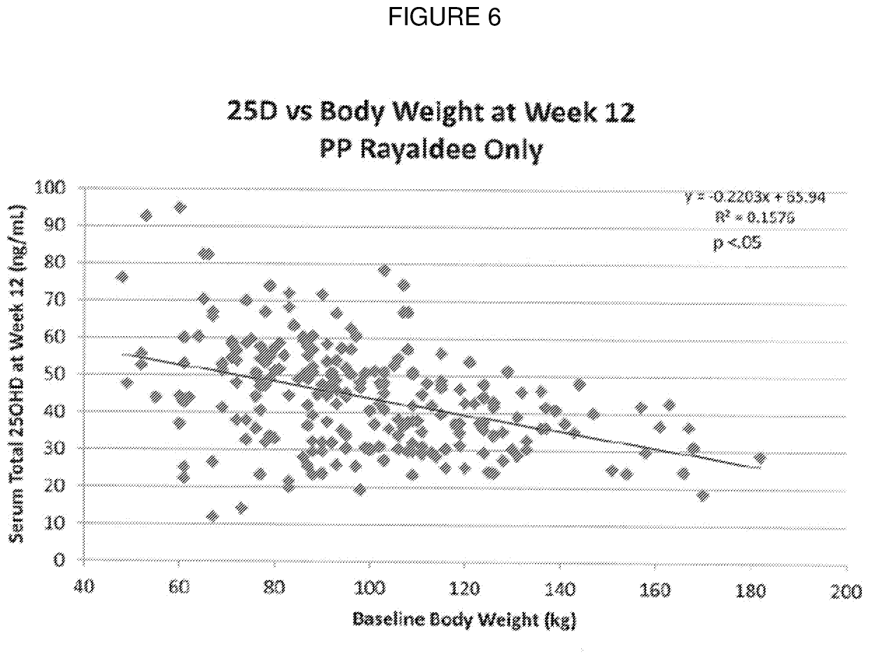

It was observed (see FIG. 5 from Example 1 below) that after 12 weeks of daily dosing with 30 mcg Rayaldee® extended release calcifediol, that patients having lower body weight at initiation of therapy experienced relatively greater rises in serum 25-hydroxyvitamin D, while patients having relatively higher body weight at initiation of therapy experienced relatively lower rises in serum 25-hydroxyvitamin D. The patient's resulting serum 25-hydroxyvitamin D concentrations after the 12 weeks of therapy also tended to relatively higher levels for relatively lower weight patients, while the levels were also influenced by the patients' baseline serum 25-hydroxyvitamin D concentrations (see FIG. 6 from Example 1 below). In other words, patients with higher body weight require higher doses of 25-hydroxyvitamin D to experience equivalent rises in serum 25-hydroxyvitamin D. Likewise, for a high-weight patient who is vitamin D insufficient or deficient (e.g. baseline serum 25-hydroxyvitamin D of 10 ng/ml), it will take a relatively higher dose for that patient to reach a serum 25-hydroxyvitamin D level of 50 ng/ml, and also a higher dose for that patient to experience an at least 30% reduction in plasma iPTH.

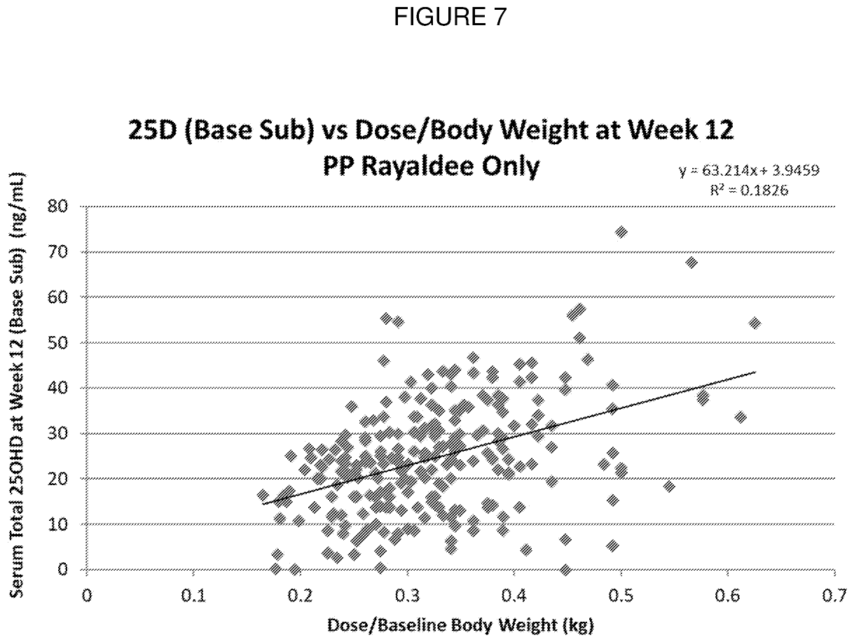

It was also observed (see FIG. 7 from Example 1 below) that the patients' increase in serum 25-hydroxyvitamin D concentrations (ng/ml), as a function of dose (30 mcg) per baseline body weight (kg) showed a positive correlation, e.g. when fit with a linear model had a slope of about 63, or about 70 if adjusted to a zero intercept.

In view of the foregoing, provided herein is a method of treating hyperparathyroidism (e.g. SHPT) in a patient (e.g. a patient having CKD) including administering to the patient a dose of 25-hydroxyvitamin D selected based on the patient's weight and baseline serum 25-hydroxyvitamin D concentration, or based on the patient's weight and desired rise in serum 25-hydroxyvitamin D. Also provided is a method of treating any condition which would benefit from increased serum 25-hydroxyvitamin D concentration (e.g. vitamin D insufficiency) including administering to the patient a dose of 25-hydroxyvitamin D selected based on the patient's weight and baseline serum 25-hydroxyvitamin D concentration, or based on the patient's weight and desired rise in serum 25-hydroxyvitamin D. The dose can be selected to provide any desired rise in serum 25-hydroxyvitamin D concentration (e.g. at least 10 ng/ml, or 20 ng/ml, or 30 ng/ml, or 40 ng/ml, or 45 ng/ml, or 50 ng/ml) or any post-treatment (or steady state) serum 25-hydroxyvitamin D concentration (e.g. at least 50 ng/ml, or at least 50.8 ng/ml, or at least 51 ng/ml, or at least 60 ng/ml). The administration can be by any suitable form and route of administration, e.g. extended release dosing by any route, e.g. oral dosing by any release mechanism, and is also contemplated as extended release oral dosing. The frequency of administration can be selected as desired, e.g. daily, every other day, thrice weekly, weekly, biweekly, or monthly. Daily dosing by extended release oral dosing is contemplated. If a frequency other than daily dosing is selected, the dose can be simply scaled (ratioed) based on an equivalent daily dosing concentration, e.g. 210 mcg weekly instead of 30 mcg daily.

In one type of embodiment, the dose (D) in mcg is a daily dose or equivalent to a daily dose selected as a function of the patient's body weight at initiation of therapy (W) in kilograms and desired rise in serum 25-hyroxyvitamin D (R) in ng/ml with scaling factor (F) according to the relationship D=(R×W)/F, wherein F is in a range of about 50 to about 80, or about 55 to about 80, or about 60 to about 75, or about 68 to about 72, or about 69 to about 71, or about 70. For example, a 70 kg patient requiring a 40 ng/ml rise in serum 25-hydroxyvitamin D can be given a 40 mcg daily dose, and a 120 kg patient requiring a 40 ng/ml rise in serum 25-hydroxyvitamin D can be given a 70 mcg daily dose. In another type of embodiment, the scaling factor F can have an additional component based on the patient's weight at initiation of therapy, e.g. such that a patient having a higher weight gets a relatively higher mcg/kg dose compared to a patient having a relatively lower weight (e.g. F=f−(Y×W)), wherein f is in a range of about 60 to about 80, or about 65 to about 75, or about 68 to about 72, or about 69 to about 71, or about 70, and Y is a unitless adjustment factor in a range of 0.01 to 0.1).

The foregoing dose selections can be used in any other method which is an aspect of the disclosure herein, optionally in combination with other dosing and outcome elements described herein.

In any method or use according to the disclosure herein, effective administration of 25-hydroxyvitamin D can include avoiding disturbing calcium metabolism, or phosphorous metabolism, or a marker thereof, or any combination of the foregoing. For example, the method can include not significantly increasing serum calcium, or not significantly increasing serum phosphorous, or not significantly increasing FGF23, with respect to pre-treatment baseline concentrations. The method can include not significantly increasing serum calcium and not significantly increasing serum phosphorous, with respect to pre-treatment baseline concentrations. The method can include not significantly increasing serum calcium, not significantly increasing serum phosphorous, and not significantly increasing FGF23, with respect to pre-treatment baseline concentrations. The method can avoid causing hypercalcemia, or avoid causing hyperphosphatemia, or avoid causing hypercalciuria, or avoid elevating FGF23 with respect to pre-treatment baseline concentration. For example, the method can include not causing hypercalcemia and hyperphosphatemia. The method can include not causing hypercalcemia, hyperphosphatemia and elevated FGF23 with respect to pre-treatment baseline concentration. The method can include not causing hypercalcemia, hyperphosphatemia, hypercalciuria, and elevated FGF23 with respect to pre-treatment baseline concentrations.

In any method or use according to the disclosure herein, effective administration of 25-hydroxyvitamin D can include providing a relatively low mean daily rise in serum total 25-hydroxyvitamin D during increase of serum total 25-hydroxyvitamin D to a steady state target level, e.g. a mean daily rise of 4 ng/mL or less, or 3.5 ng/mL or less, or 3 ng/mL or less, or 2 ng/mL or less. Optionally, the average daily rise in serum total 25-hydroxyvitamin D during increase of serum total 25-hydroxyvitamin D can be at least 0.2 ng/mL, or at least 0.3 ng/mL, or at least 0.5 ng/mL, or at least 1 ng/mL, or at least 2 ng/mL, or at least 2.5 ng/mL, for example in a range of about 0.2 ng/mL to about 4 ng/mL, or about 0.2 ng/mL to about 3.5 ng/mL, or about 0.2 ng/mL to about 3 ng/mL, or about 0.2 ng/mL to about 2.5 ng/mL, or about 0.2 ng/mL to about 2 ng/mL, or about 0.2 ng/mL to about 1 ng/mL, or about 0.3 ng/mL to about 4 ng/mL, or about 0.3 ng/mL to about 3.5 ng/mL, or about 0.3 ng/mL to about 3 ng/mL, or about 0.3 ng/mL to about 2.5 ng/mL, or about 0.3 ng/mL to about 2 ng/mL, or about 0.3 ng/mL to about 1 ng/mL. An upper limit of about 3 ng/mL is particularly contemplated. Similarly, the maximum serum total 25-hydroxyvitamin D rise within a 24 hour period following an individual dose (ΔC24) can be 4 ng/mL or less, or 3.5 ng/mL or less, or 3 ng/mL or less, or 2 ng/mL or less, and optionally at least 0.2 ng/mL, or at least 0.3 ng/mL, or at least 0.5 ng/mL, or at least 1 ng/mL, or at least 2 ng/mL, or at least 2.5 ng/mL, for example in a range of about 0.2 ng/mL to about 4 ng/mL, or about 0.2 ng/mL to about 3.5 ng/mL, or about 0.2 ng/mL to about 3 ng/mL, or about 0.2 ng/mL to about 2.5 ng/mL, or about 0.2 ng/mL to about 2 ng/mL, or about 0.2 ng/mL to about 1 ng/mL, or about 0.3 ng/mL to about 4 ng/mL, or about 0.3 ng/mL to about 3.5 ng/mL, or about 0.3 ng/mL to about 3 ng/mL, or about 0.3 ng/mL to about 2.5 ng/mL, or about 0.3 ng/mL to about 2 ng/mL, or about 0.3 ng/mL to about 1 ng/mL. An upper limit of about 3 ng/mL is particularly contemplated.

In an alternative method according to the disclosure herein, the method can include providing a relatively low mean daily rise in serum total 25-hydroxyvitamin D during increase of serum total 25-hydroxyvitamin D to a steady state target level, e.g. of 4 ng/mL or less, or 3 ng/mL or less, or 2 ng/mL or less, and optionally at least 0.2 ng/mL, or at least 0.3 ng/mL while providing a ΔC24 that can be in excess of 3 ng/mL, e.g. at least 0.2 ng/mL, 0.3 ng/mL, 1 ng/mL, 2 ng/mL, 3 ng/mL, 5 ng/mL, or 10 ng/mL and up to 30 ng/mL, or 20 ng/mL, or 10 ng/mL, for example in a range of about 0.2 ng/mL to 30 ng/mL, or 0.3 ng/mL to 10 ng/mL, or 0.3 ng/mL to 20 ng/mL, or >3 ng/mL to 30 ng/mL, >3 ng/mL to 20 ng/mL, or >3 ng/mL to 10 ng/mL, or >3 ng/mL to 7 ng/mL, or >3 ng/mL to <7 ng/mL, or >3 ng/mL to 6 ng/mL, or >3 ng/mL to 5 ng/mL, or >3 ng/mL to 4 ng/mL, optionally through long-frequency dosing, e.g. monthly, biweekly, or weekly, for example.

In another alternative method according to the disclosure herein, the method can include providing an mean daily rise in serum total 25-hydroxyvitamin D during increase of serum total 25-hydroxyvitamin D to a steady state target level wherein the rise is a range of about 0.2 ng/mL to about 10 ng/mL, or about 0.3 ng/mL to about 10 ng/mL, or about 0.5 ng/mL to about 10 ng/mL, or about 1 ng/mL to about 10 ng/mL to about e.g. of 3 ng/mL or less or 2 ng/mL or less, and optionally at least 0.2 ng/mL, or at least 0.3 ng/mL while providing a ΔC24 that can be at least 0.2 ng/mL, 0.3 ng/mL, 1 ng/mL, 2 ng/mL, 3 ng/mL, 5 ng/mL, or 10 ng/mL and up to 30 ng/mL, or 20 ng/mL, or 10 ng/mL, for example in a range of about 0.2 ng/mL to 30 ng/mL, or 0.3 ng/mL to 10 ng/mL, or 0.3 ng/mL to 20 ng/mL, or >3 ng/mL to 30 ng/mL, >3 ng/mL to 20 ng/mL, or >3 ng/mL to 10 ng/mL, or >3 ng/mL to 7 ng/mL, or >3 ng/mL to <7 ng/mL, or >3 ng/mL to 6 ng/mL, or >3 ng/mL to 5 ng/mL, or >3 ng/mL to 4 ng/mL, optionally through long-frequency dosing, e.g. monthly, biweekly, or weekly, for example.

In any method or use according to the disclosure herein, effective administration of 25-hydroxyvitamin D can include increasing serum total 25-hydroxyvitamin D to a steady state level over a period of at least 8 weeks, or at least 10 weeks, or at least 12 weeks, or at least 14 weeks, for example over a period of 8 to 14 weeks, or 8 to 12 weeks, or 10 to 12 weeks. For example, effective administration of 25-hydroxyvitamin D can include increasing serum total 25-hydroxyvitamin D to a steady state level in a range of about 50 to about 300 ng/mL, or about 50 to about 200 ng/mL, or about 50 to about 100 ng/mL, or greater than 50 to about 300 ng/mL, or greater than 50 to about 200 ng/mL, or greater than 50 to about 100 ng/mL, optionally at least 50.8 ng/mL, optionally at least 51 ng/mL, optionally, in a range of about 60 to about 300 ng/mL, or about 60 to about 200 ng/mL, or about 60 to about 100 ng/mL, or greater than 60 to about 300 ng/mL, or greater than 60 to about 200 ng/mL, or greater than 60 to about 100 ng/mL, over a period of at least 8 weeks, or at least 10 weeks, or at least 12 weeks, or at least 14 weeks, for example over a period of 8 to 14 weeks, or 8 to 12 weeks, or 10 to 12 weeks.

In any method or use according to the disclosure herein, effective administration of 25-hydroxyvitamin D can include administering 25-hydroxyvitamin D to increase the patient's serum total 1,25-dihydroxyvitamin D to a steady state level of at least 40 pg/mL, or at least 45 pg/mL, and optionally not more than 62 pg/mL, for example in a range of 40 pg/mL or 45 pg/mL.

In any method or use according to the disclosure herein, the patient can be one who is vitamin D insufficient at initiation of therapy, e.g. having serum total 25-hydroxyvitamin D less than 30 ng/mL. Optionally, the patient's serum total 25-hydroxyvitamin D at the initiation of therapy can be less than 30 ng/mL.

In any method or use according to the disclosure herein, the patient can be one who has a serum total 25-hydroxyvitamin D greater than or about 30 ng/mL at initiation of therapy. Optionally, the patient can be one who has a serum total 25-hydroxyvitamin D greater than or about 40 ng/mL at initiation of therapy.

In any method or use according to the disclosure herein, the patient can be one who has CKD, optionally CKD Stage 3 to 5, or Stage 3 to 4, or Stage 5. Optionally, the patient can be one who is also being treated by hemodialysis.

The method can include 25-hydroxyvitamin D therapy to reduce plasma iPTH level. The method can include 25-hydroxyvitamin D to reduce plasma iPTH by at least about 15%, for example, at least about 15%, at least about 20%, at least about 25%, at least about 30%, at least about 35%, at least about 40%, at least about 45%, or at least about 50%, compared to its pre-treatment level. In another aspect, repeat doses of 25-hydroxyvitamin D are optionally administered to a patient population in an amount effective to lower the mean plasma intact PTH level of the patient population by at least about 15%, for example, at least about 15%, at least about 20%, at least about 25%, at least about 30%, at least about 35%, at least about 40%, at least about 45%, or at least about 50%, compared to its pre-treatment level.

The method can include 25-hydroxyvitamin D therapy to increase bone mineral density, e.g. to T-score of at least −2.5, or greater than −2.5, or at least −2.0, or at least −1.5, or at least −1.0 or greater than −1.0. The method can include 25-hydroxyvitamin D therapy to decrease the blood level of a bone resorption marker, e.g. one or more of serum total alkaline phosphatase, BSAP, CTX-1, P1NP, and FGF-23. For example, the marker can be reduced to within the reference range for the laboratory measurement technique. In another aspect, the marker can be reduced by at least about 10%, or at least about 20%, or at least about 30%.

In another aspect, the method can include administering 25-hydroxyvitamin D therapy as described herein and in the absence of 1,25-dihydroxyvitamin D therapy, or in the absence of calcitriol therapy, or in the absence of doxercalciferol therapy, or in the absence of alfacalcidol therapy, or in the absence of paricalcitol therapy, or in the absence of maxacalcitol therapy, or in the absence of falecalcitriol therapy, or in the absence of therapy with an active vitamin D analog.

In another aspect, the method can include administering 25-hydroxyvitamin D therapy as described herein and in the absence of cinacalcet therapy.

In another aspect, the method can include administering 25-hydroxyvitamin D therapy as described herein and co-administering cinacalcet therapy.

While the 25-hydroxyvitamin D can be administered in any form, in one aspect the 25-hydroxyvitamin D can be administered by modified release, for example by sustained release or by delayed-sustained release. For example, the sustained release can be effected via an oral dosage form, or the sustained release can be effected via a transdermal patch. In another aspect, sustained delivery can be provided via slow injection or infusion of the compound over time, e.g. a slow push intravenous delivery. For example, intravenous delivery can be over a period of time of at least one hour, optionally over one hour to five hours. The administration can be concomitant with hemodialysis treatment, for example. In an alternative aspect, the administration can be while the patient is not receiving hemodialysis.

In one type of embodiment, the 25-hydroxyvitamin D is administered orally. For example, the 25-hydroxyvitamin D can be administered in an oral sustained release formulation. In the alternative, the 25-hydroxyvitamin D can be administered in an oral immediate release formulation in multiple doses over an extended time period throughout a day, in order to produce a pharmacokinetic profile of serum 25-hydroxyvitamin D that is similar to that achieved by an oral sustained release formulation.

In any method or use according to the disclosure herein, effective administration of 25-hydroxyvitamin D can include administering 25-hydroxyvitamin D to avoid substantial induction of CYP24A1. For example, the method can include administering 25-hydroxyvitamin D to achieve a vitamin D metabolite ratio (VMR), calculated as serum 24,25-dihydroxyvitamin D₃/serum total 25-hydroxyvitamin D₃*100, of 5 or less, or 4.8 or less.

Hyperparathyroidism can be caused by chronically low serum calcium levels, vitamin D deficiency, and kidney disease. Hyperparathyroidism also can be caused by a benign tumor (adenoma) of a parathyroid gland, or less frequently a cancerous tumor. Hyperparathyroidism can also be caused when two or more parathyroid glands become enlarged (hyperplasia). Hyperparathyroidism can be caused by other dysfunctions of parathyroid glands, including hypertrophy of the parathyroid gland, multiple endocrine neoplasia, exposure to radiation, and use of lithium therapy. Parathyroid gland neoplasias giving rise to hyperparathyroidism include multiple endocrine neoplasias MEN1 and MEN2A.

Diseases and conditions associated with an increase in plasma iPTH over normal baseline values include renal osteodystrophy, osteitis fibrosa cystica, osteomalacia, osteoporosis, osteopenia, extraskeletal calcification and related disorders, e.g., bone pain, periarticular inflammation and Mockerberg's sclerosis. Soft-tissue and vascular calcification, including pulmonary vascular calcification and pulmonary hypertension, cardiovascular disease, and calcific uremic arteriolopathy (CUA) are additional serious consequences of hyperparathyroidism. Other manifestations of hyperparathyroidism include alterations in cardiovascular structure and function. immune dysfunction, renal anaemia, neurological disturbances, hematological abnormalities, and endocrine dysfunction. Hyperparathyroidism can be associated with Aging-Related Vitamin D Deficiency (ARVDD) syndrome

A controlled release composition intended for oral can be designed to contain concentrations of 25-hydroxyvitamin D₂ and/or 25-hydroxyvitamin D₃ of 1 to 1000 μg per unit dose, for example, and prepared in such a manner as to effect substantially constant release of the 25-hydroxyvitamin D₂ and/or 25-hydroxyvitamin D₃ over an extended period of time, e.g. at least 4 hours, or at least 8 hours, or at least 12 hours, or at least 24 hours.

The preparation of a sustained release form of 25-hydroxyvitamin D suitable for oral administration can be carried out according to many different techniques. For example, one or more 25-hydroxyvitamin D compounds can be dispersed within a matrix, i.e., a chosen mixture of rate controlling constituents and excipients in selected ratios within the matrix, and optionally encased with a coating material. In another alternative, one or more of various coating techniques can be utilized to control the rate of the release of the 25-hydroxyvitamin D from the pharmaceutical formulation. For example, a gradual dissolution of a coating over time can expose the dosage form contents, optionally in a matrix, to the fluid of the local environment. In one type of embodiment, after a coating becomes permeable, 25-hydroxyvitamin D diffuses through the coating, e.g. from the outer surface of the matrix contained within the coating. When the surface of such a matrix becomes exhausted or depleted of 25-hydroxyvitamin D, the underlying stores begin to be depleted by diffusion through the matrix to the external solution. In another type of embodiment, release of 25-hydroxyvitamin D through a permeable coating or framework is influenced gradual disintegration or erosion of a matrix contained therein, e.g., via solubility of one or more components of the matrix. In another type of embodiment, release of 25-hydroxyvitamin D is by gradual disintegration or erosion of a matrix, e.g., via solubility of one or more components of the matrix and/or by lack of physical integrity, without any coating or other framework surrounding the matrix. The dosage form can optionally further comprise another active agent, in the same region or a different region from the 25-hydroxyvitamin D. For example, the additional active agent can include calcium.

In one aspect, a formulation provides one or more 25-hydroxyvitamin D compounds within a matrix that releasably binds the ingredients for sustained release, e.g., when exposed to the contents of the gastric tract, e.g. stomach, small intestine, or colon.

In one embodiment of the invention, a controlled release oral formulation of 25-hydroxyvitamin D is prepared generally according to the following procedure. A sufficient quantity of 25-hydroxyvitamin D, e.g. calcifediol, is completely dissolved in a minimal volume of USP-grade absolute ethanol (or other suitable solvent) and mixed with appropriate amounts and types of pharmaceutical-grade excipients to form a matrix which is solid or semi-solid at both room temperature and at the normal temperature of the human body. The matrix gradually disintegrates in the intestine and/or colon.

In a suitable formulation, the matrix binds the 25-hydroxyvitamin D compound(s) and permits a slow, relatively steady, e.g. substantially constant, release of 25-hydroxyvitamin D over a period of four to eight hours or more, by simple diffusion and/or gradual disintegration, into the contents of small intestine and/or colon.

As discussed above, the means for providing the controlled release of 25-hydroxyvitamin D may be selected from any suitable controlled release delivery system, including any of the known controlled release delivery systems of an active ingredient over a course of about four or more hours, including the wax matrix system, and the EUDRAGIT RS/RL system (Rohm Pharma, GmbH, Weiterstadt, Germany).

The wax matrix system provides one type of a lipophilic matrix. The wax matrix system may utilize, for example, beeswax, white wax, cachalot wax or similar compositions. In one type of embodiment, the wax is a non-digestible wax, e.g. paraffin. The active ingredient(s) are dispersed in the wax binder which slowly disintegrates in intestinal fluids to gradually release the active ingredient(s). The wax binder that is impregnated with 25-hydroxyvitamin D can be loaded into softgel capsules. A softgel capsule may comprise one or more gel-forming agents, e.g., gelatin, starch, carrageenan, and/or other pharmaceutically acceptable polymers. In one embodiment, partially crosslinked soft gelatin capsules are used. As another option, vegetable-based capsules can be used. The wax matrix system disperses the active ingredient(s) in a wax binder which softens at body temperature and slowly disintegrates in intestinal fluids to gradually release the active ingredient(s). The system suitably can include a mixture of waxes, with the optional addition of oils, to achieve a melting point which is higher than body temperature, but lower than the melting temperature of the selected formulations used to create the shell of a soft or hard capsule, or vegetable capsule shell, or other formulation used to create a shell casing or other coating.

Specifically, in one suitable embodiment, the waxes selected for the matrix are melted and thoroughly mixed. The desired quantity of oils is subsequently added, followed by sufficient mixing for homogenization. The waxy mixture is then gradually cooled to a temperature just above its melting point. The desired amount of 25-hydroxyvitamin D, dissolved in ethanol, is uniformly distributed into the molten matrix, and the matrix is loaded into capsules, for example vegetable-based or gelatin-based capsules. The filled capsules optionally are treated for appropriate periods of time with a solution containing an aldehyde, such as acetaldehyde, to partially crosslink a polymer, e.g., gelatin, in the capsule shell, when used. The capsule shell becomes increasingly crosslinked, over a period of several weeks and, thereby, more resistant to dissolution in the contents of stomach and upper intestine. When properly constructed, this gelatin shell will gradually dissolve after oral administration and become sufficiently porous (without fully disintegrating) by the time it reaches the small intestine, to allow the 25-hydroxyvitamin D to diffuse slowly from the wax matrix into the contents of the small intestine and/or colon.

Examples of other lipid matrices suitable for use with the methods of the invention include one or more of glycerides, fatty acids and alcohols, and fatty acid esters.

A wax matrix can contain a stabilizing component to stabilize the release properties of the dosage form over its expected shelf life. The stabilizing component can be a cellulosic component, for example a cellulose ether, e.g. hydroxyl propyl methylcellulose.

In one embodiment, a formulation may comprise an oily vehicle for the 25-hydroxyvitamin D compound. Any pharmaceutically-acceptable oil can be used. Examples include animal (e.g., fish), vegetable (e.g., soybean), and mineral oils. The oil preferably will readily dissolve the 25-hydroxyvitamin D compound used. Oily vehicles can include non-digestible oils, such as mineral oils, particularly liquid paraffins, and squalene. The ratio between the wax matrix and the oily vehicle can be optimized in order to achieve the desired rate of release of the 25-hydroxyvitamin D compound. Thus, if a heavier oil component is used, relatively less of the wax matrix can be used, and if a lighter oil component is used, then relatively more wax matrix can be used.

Another suitable controlled-release oral drug delivery system is the EUDRAGIT RL/RS system in which the active 25-hydroxyvitamin D ingredient is formed into granules, e.g. having a dimension of 25/30 mesh. The granules are then uniformly coated with a thin polymeric lacquer, which is water-insoluble but slowly water-permeable. The coated granules can be mixed with optional additives including one or more of antioxidants, stabilizers, binders, lubricants, processing aids and the like. The mixture may be compacted into a tablet which, prior to use, is hard and dry and can be further coated, or it may be poured into a capsule. After the tablet or capsule is swallowed and comes into contact with the aqueous gastric and intestinal fluids, the thin lacquer begins to swell and slowly allows permeation by intestinal fluids. As the intestinal fluid slowly permeates the lacquer coating, the contained 25-hydroxyvitamin D is slowly released. By the time the tablet or capsule has passed through the small intestine, about four to eight hours or more later, the 25-hydroxyvitamin D will have been slowly, but completely, released. Accordingly, the ingested tablet will release a stream of 25-hydroxyvitamin D, as well as any other active ingredient.

The EUDRAGIT system is comprised of high permeability lacquers (RL) and low permeability lacquers (RS). RS is a water-insoluble film former based on neutral swellable methacrylic acids esters with a small proportion of trimethylammonioethyl methacrylate chlorides; the molar ratio of the quaternary ammonium groups to the neutral ester group is about 1:40. RL is also a water insoluble swellable film former based on neutral methacrylic acid esters with a small portion of trimethylammonioethyl methacrylate chloride, the molar ratio of quaternary ammonium groups to neutral ester groups is about 1:20. The permeability of the coating and thus the time course of drug release can be titrated by varying the proportion of RS to RL coating material. For further details of the Eudragit RL/RS system, reference is made to technical publications available from Rohm Tech, Inc. 195 Canal Street, Maiden, Mass., 02146 and K. Lehmann, D. Dreher “Coating of tablets and small particles with acrylic resins by fluid bed technology,” Int. J. Pharm. Tech. & Prod. Mfr. 2(r), 31-43 (1981), incorporated herein by reference.

Other examples of insoluble polymers include polyvinyl esters, polyvinyl acetals, polyacrylic acid esters, butadiene styrene copolymers and the like.

The dosage forms may also contain adjuvants, such as preserving or stabilizing adjuvants. For example, a preferred formulation includes 25-hydroxyvitamin D (e.g., about 30 μg, about 60 μg, or about 90 μg 25-hydroxyvitamin D₃), about 2 wt % anhydrous ethanol, about 10 wt % lauroyl polyoxylglycerides, about 20 wt % hard paraffin, about 23 wt % glycerol monostearate, about 35 wt % liquid paraffin or mineral oil, about 10 wt % hydroxypropyl methylcellulose, and optionally a small amount of antioxidant preservative (e.g., butylated hydroxytoluene). Formulations according to the invention may also contain other therapeutically valuable substances or may contain more than one of the compounds specified herein and in the claims in admixture.

As an alternative to oral 25-hydroxyvitamin D, intravenous administration of 25-hydroxyvitamin D is also contemplated. In one embodiment, the 25-hydroxyvitamin D is administered as a sterile intravenous bolus, optionally a bolus injection of a composition that results in a sustained release profile. In another embodiment, the 25-hydroxyvitamin D is administered via gradual injection/infusion, e.g., over a period of 1 to 5 hours, to effect controlled or substantially constant release of the 25-hydroxyvitamin D directly to DBP in the blood of the patient. For example, the composition may be injected or infused over a course of at least about 1 hour, at least about 2 hours, at least about 3 hours, at least about 4 hours, at least about 5 hours, or at least about 6 hours. In one embodiment, the composition intended for intravenous administration in accordance with the present invention is designed to contain a concentration of the 25-hydroxyvitamin D compound(s) of 1 to 100 μg per unit dose. Sterile, isotonic formulations of 25-hydroxyvitamin D may be prepared by dissolving 25-hydroxyvitamin D in absolute ethanol, propylene glycol or another suitable solvent, and combining the resulting solution with one or more surfactants, salts and preservatives in appropriate volumes of water for injection. Such formulations can be administered slowly from syringes, for example, via heparin locks, or by addition to larger volumes of sterile solutions (e.g., saline solution) being steadily infused over time.

Suitable sustained release dosage forms of 25-hydroxyvitamin D have been described, including in the following US patent and patent application publications, the disclosures of which are hereby incorporated by reference herein: 2010/0120728A1, 2010/0144684A1, 2013/0137663A1, 8,329,677, 8,361,488, 8,426,391, 8,962,239, and 9,861,644.

The following examples are given merely to illustrate the present invention and not in any way to limit its scope.

### EXAMPLES

The following examples are provided for illustration and are not intended to limit the scope of the invention.

### Example 1

Adult subjects (n=429) with SHPT, VDI and stage 3 or 4 CKD were stratified by stage and treated daily with either extended-release calcifediol (ERC) or placebo in two identical, parallel, randomized, double-blind studies. After treatment for 26 weeks, all subjects were ranked by the level of serum total 25-hydroxyvitamin D and divided into quintiles in order to examine the relationships between the degree of vitamin D repletion and the associated changes in plasma iPTH, serum bone turnover markers, calcium, phosphorus, intact fibroblast growth factor 23 (FGF23) and vitamin D metabolites, estimated glomerular filtration rate (eGFR) and urine calcium-to-creatinine (Ca:Cr) ratio.

Specifically, two identical 26-week multicenter studies with randomized, double-blind, placebo-controlled designs enrolled a total of 429 subjects from 89 US sites with SHPT (plasma iPTH≥85 and <500 pg/mL), stage 3 or 4 CKD (eGFR of ≥15 and <60 mL/min/1 73m2), and VDI (serum total 25-hydroxyvitamin D≥10 and <30 ng/mL). Other eligibility criteria included serum calcium≥8.4 and <9.8 mg/dL and serum phosphorus≥2.0 and <5.0 mg/dL. Exclusion criteria included a spot urine calcium:creatinine (Ca:Cr) ratio of >0.2, nephrotic range proteinuria (>3 mg/mg Cr) and history of parathyroidectomy for SHPT or renal transplantation. Subjects were enrolled progressively at sites of many different latitudes in order to minimize seasonal variation in mean baseline serum total 25-hydroxyvitamin D. Further details regarding these studies have been previously published in Sprague et al., Use of extended-release calcifediol to treat secondary hyperparathyroidism in stages 3 and 4 chronic kidney disease, Am J Nephrol 2016; 44:316-325, and Sprague et al., Extended-release calcifediol for secondary hyperparathyroidism in stage 3-4 chronic kidney disease, Expert Review of Endocrinology & Metabolism 2017; 12:289-301, the disclosures of which are incorporated herein by reference.

Subjects were stratified by CKD stage and were randomized in a 2:1 ratio to receive a once daily 30 μg oral dose of ERC (or matching placebo) for 12 weeks at bedtime followed by an additional 14 weeks of treatment with once daily bedtime doses of either 30 or 60 μg of ERC (or placebo). The daily dose was increased to 60 μg at the start of week 13 if plasma iPTH remained >70 pg/mL (the upper limit of the laboratory reference range), serum total 25-hydroxyvitamin D was <65 ng/mL (to reduce the risk of driving values above 100 ng/mL) and serum calcium was <9.8 mg/dL. The sole primary efficacy end point was the proportion of subjects in the intent-to-treat (ITT) population that attained a mean decrease of ≥30% in plasma iPTH from pre-treatment baseline in the efficacy assessment period (EAP), defined as treatment weeks 20 through 26.

A total of 213 subjects participated in the first of these two RCTs (141 ERC and 72 placebo) and 216 subjects in the other (144 ERC and 72 placebo), and 354 subjects (83%) completed the studies. Data from both RCTs were pooled because: (a) the studies were governed by a common protocol; (b) they were conducted contemporaneously using multiple sites within the continental US; (c) the subject populations were similar according to selection criteria and actual baseline demographic and biochemical characteristics; and, (d) the changes observed in serum total 25-hydroxyvitamin D, serum total 1,25-dihydroxyvitamin D and plasma iPTH were similar during ERC or placebo treatment. In aggregate, 222 subjects (51.7%) had stage 3 CKD (151 ERC and 71 placebo) and 207 subjects (48.3%) had stage 4 CKD (134 ERC and 73 placebo).

The ITT population included all subjects (n=429) who were randomized to study drug. Subjects in the ITT population had a mean age of 66 years (range 25-85), 50% were male, 65% White, 32% African-American or Black, 21% Hispanic and 3% Other. The most common causes of CKD were diabetes and hypertension and the mean eGFR was 31 mL/min/1.73 m².

The per-protocol (PP) population included all subjects (n=356) who did not have a major protocol deviation and for whom at least two serum total 25-hydroxyvitamin D and two plasma iPTH determinations were included in the calculated baseline value and in the EAP, defined as treatment weeks 20 through 26. Demographic and baseline data for the PP population are summarized in Table 1, grouped by CKD stage. Only analyses of the PP population are reported here as they yielded results that did not differ materially from those based on analyses of the ITT population, and because the number of subjects remained constant across the 26-week treatment period. Sixty-two ITT subjects were excluded because they discontinued treatment prior to the EAP, and 11 for major protocol violations: receipt of prohibited concomitant medication (n=4); failure to meet all selection criteria (n=3); dosing compliance <80% (n=3); and, premature unblinding (n=1).

TABLE 1

| | CKD 3 | CKD 4 | Total |
|---|---|---|---|
| Number of subjects | 185 | 171 | 356 |
| Male | 98 | 91 | 189 |
| Female | 87 | 80 | 167 |

Mean (SE)

| | | | |
|---|---|---|---|
| Baseline eGFR | 38.5 (0.6) | 23.7 (0.4)³ | 31.4 (0.5) |
| Weight (kg) | 98.7 (1.8) | 96.8 (1.9) | 97.8 (1.3) |
| BMI | 35.1 (0.6) | 34.2 (0.6) | 34.7 (0.4) |
| Age (years) | 65.4 (0.8) | 65.3 (0.9) | 65.4 (0.6) |
| Serum total 25(OH)D | 19.9 (0.4) | 19.2 (0.4) | 19.6 (0.3) |
| (ng/mL) | | | |
| Serum total | 39.7 (1.0) | 29.9 (1.0)³ | 34.5 (0.7) |
| 1,25(OH)2D (pg/mL) | | | |
| Serum 24,25(OH)2D₃ | 1.09 (0.04) | 1.02 (0.03) | 1.05 (0.02) |
| (ng/mL) | | | |
| Plasma iPTH (pg/mL) | 129.5 (3.0) | 160.1 (4.9)³ | 144.2 (2.9) |
| Serum total alkaline | 92.4 (2.1) | 94.2 (2.5) | 93.2 (1.6) |
| phosphatase (U/L) | | | |
| Serum BSAP (U/L) | 36.8 (1.3) | 39.3 (1.5) | 38.0 (1.0) |
| Serum CTx-1 (pg/mL) | 602 (24) | 841 (32)³ | 717.6 (20.8) |
| Serum P1NP (ng/mL) | 86.1 (3.5) | 110.5 (5.5)² | 98.0 (3.3) |
| Serum calcium (mg/dL) | 9.3 (0.03) | 9.2 (0.03)¹ | 9.3 (0.02) |
| Serum phosphorus | 3.6 (0.04) | 3.9 (0.04)³ | 3.7 (0.03) |
| (mg/dL) | | | |
| Serum FGF-23 (pg/mL) | 38.2 (3.2) | 42.0 (4.0) | 40.3 (2.6) |
| Urine Ca/Cr ratio | 0.046 (0.005) | 0.031 (0.003)¹ | 0.039 (0.003) |

¹Significantly different from CKD 3 subjects, p < 0.05
²Significantly different from CKD 3 subjects, p < 0.001
³Significantly different from CKD 3 subjects, p < 0.0001

Blood and spot urine samples were collected at weekly or biweekly intervals and analyzed during the applicable stability windows (documented in validation reports) at OOD Global Central Labs (Highland Heights, Ky.). Plasma iPTH levels were determined by two-site sandwich electrochemiluminescence (Roche Elecsys; reference range 15-65 pg/mL; % CV 2.7). Serum total 25-hydroxyvitamin D was determined by chemiluminescence (DiaSorin), and serum total 1,25-dihydroxyvitamin D was determined by radioimmunoassay (IDS). Serum 25-hydroxyvitamin D₃ (lower limit of quantitation: 5.00 ng/mL; % CV of 0.82 to 1.84 within-run, 2.01 to 4.26% between-run) and 24,25-dihydroxyvitamin D₃ (lower limit of quantitation: 0.52 ng/mL; % CV 2.18 to 4.60 within-run, 3.79 to 9.29 between-run) were determined by LC-MS (Syneos) for the purpose of calculating the vitamin D metabolite ratio (VMR), calculated as serum 24,25-dihydroxyvitamin D₃/serum total 25-hydroxyvitamin D₃*100. Serum (rather than plasma) intact FGF23 levels were determined by enzyme-linked immunosorbent assay (Millipore; reference range 0-50 pg/mL; % CV 10.6) because of better recovery and long-term stability during validation. Serum collagen type 1 C-telopeptide (CTx-1) was measured by electrochemiluminescence (Roche Cobas; reference range 0-856 pg/mL; % CV 1.4). Intact procollagen type 1 N-terminal propeptide (P1NP) was determined by chemiluminescence immunoassay (Roche Cobas; reference rang 13.8-88 ng/mL; % CV 5.0), an assay which measures monomers that potentially accumulate in CKD patients, leading to falsely elevated results. Bone-specific alkaline phosphatase (BSAP) was determined by ELISA (Quidel; reference range 14.9-42.4 U/L, % CV 7.7), an assay which measures activity rather than mass. Total alkaline phosphatase was measured by enzymatic assay (Roche Cobas; reference range 43-115 U/L; % CV 2.0). Other parameters were determined by standard procedures. Serum calcium values were corrected for low albumin.

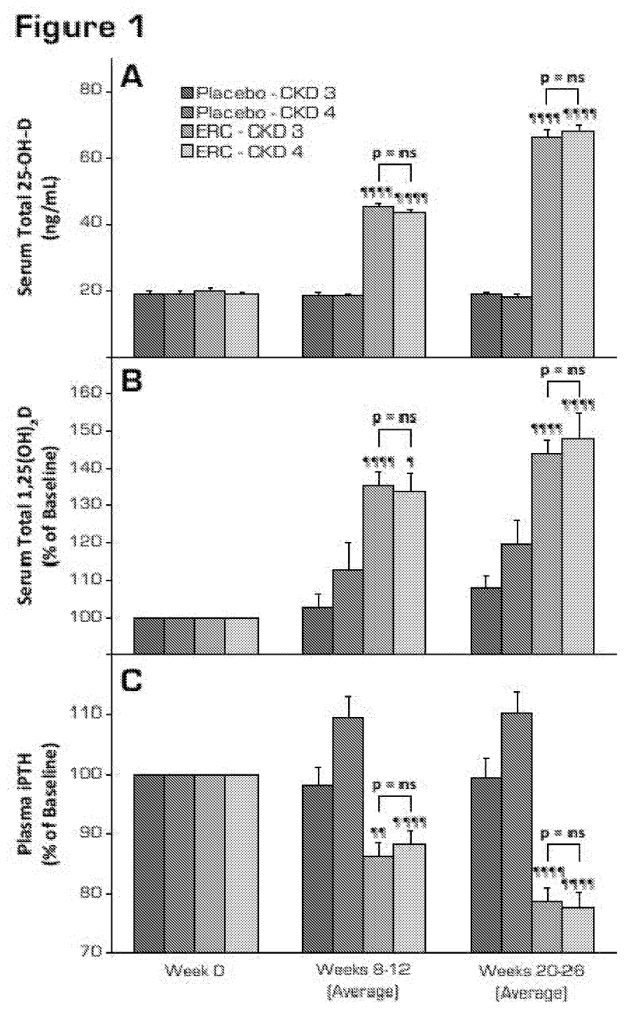

Changes from pre-treatment baseline in mean serum total 25-hydroxyvitamin D and 1,25-dihydroxyvitamin D and plasma iPTH in response to treatment with ERC or placebo were examined by CKD stage both midway through the study (at treatment weeks 8-12) and at the EAP (treatment weeks 20-26). ERC increased mean serum 25-hydroxyvitamin D similarly versus placebo (P<0.0001) in both CKD stages at mid-study and at the EAP (FIG. 1A). ERC also increased mean serum 1,25-dihydroxyvitamin D and reduced mean plasma iPTH similarly versus placebo (P<0.05 to 0.0001) in both CKD stages at mid-study and at the EAP (FIGS. 1B and 10). Values for serum 1,25-dihydroxyvitamin D and plasma iPTH were expressed as percentages of baseline since subjects with stage 3 CKD had different mean baseline values than subjects with stage 4 CKD (Table 1). Given the lack of stage-specific responses for these three key parameters, all further analyses were completed without regard to CKD stage.

Demographic and baseline data for P subjects grouped by post-treatment 25-hydroxyvitamin D quintile are shown in Table 2. Analysis of plasma iPTH and serum bone turnover markers by duration of treatment and post-treatment 2-hydroxyvitamin D quintile are shown in Table 3.

All subjects, whether treated with ERC or placebo, were subsequently ranked by mean post-treatment (EAP) serum total 25-hydroxyvitamin D levels and divided into quintiles, with Quintile 1 being defined as subjects with the lowest levels and Quintiles 2-5 as those with progressively higher levels. The mean (SE) post-treatment 25-hydroxyvitamin D values in each quintile are noted at the top of Table 2, where the demographic and baseline characteristics of the PP subjects are summarized (by quintile) and at the left in Table 3. Means for Quintiles 2-5 were all significantly greater than the corresponding mean for Quintile 1 (p<0.0001). The proportions of subjects treated with placebo in Quintiles 1 and 2 were 96% and 76%, respectively. There were no placebo subjects in Quintiles 3-5. Data from Quintiles 2-5 were compared to those from Quintile 1 by one-way ANOVA with subsequent Bonferroni's correction. Mean (SE) serum total 25-hydroxyvitamin D at baseline ranged from 16.1 (0.6) to 21.7 (0.6) ng/mL (P<0.05) within the individual quintiles. Significant variations between quintiles at baseline were also apparent for mean body weight, body mass index (BMI) and age, as noted, but no differences were detected for mean eGFR or for mean serum and urine parameters associated with mineral and bone metabolism.

Mean (SE) post-treatment serum total 1,25-dihydroxyvitamin D increased progressively across the quintiles from 34.3 (1.3) pg/mL in Quintile 1 to 48.5 (2.1) pg/mL in Quintile 5. Means for Quintiles 2-5 were all significantly greater than the mean for Quintile 1 (p<0.01).

Mean (SE) post-treatment serum 24,25-dihydroxyvitamin D₃ increased progressively from 0.7 (0.04) in Quintile 1 to 5.6 (0.27) ng/mL in Quintile 5. Values differed from Quintile 1 for only for the two highest quintiles (P<0.05).

Mean (SE) post-treatment VMR rose progressively from 3.6 (0.22) for Quintile 1 to 4.8 (0.22) in Quintile 4 but remained stable thereafter at 4.7 (0.19) in Quintile 5.

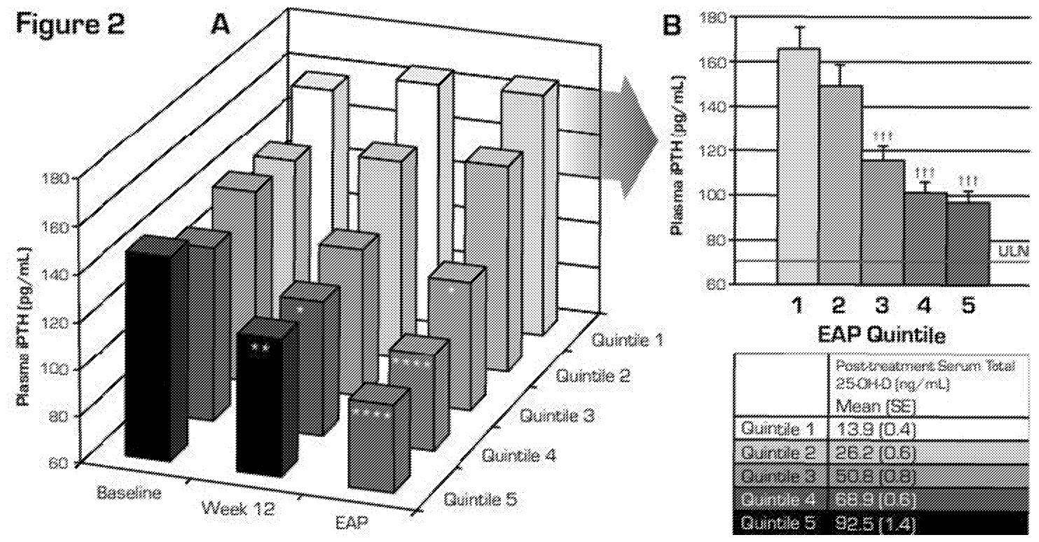

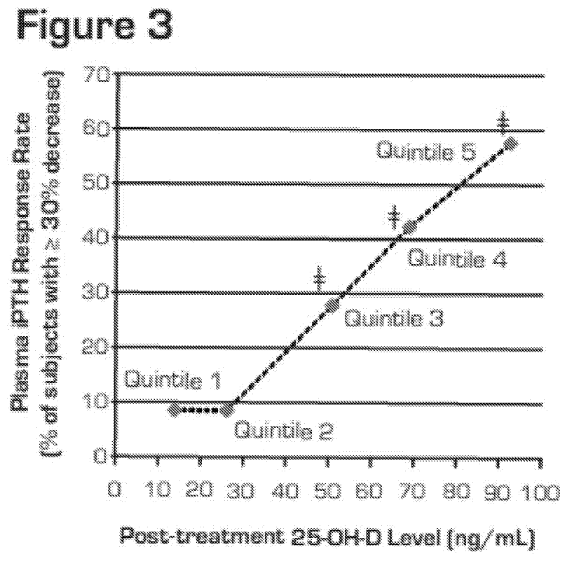

Mean (SE) plasma iPTH trended upward during treatment in Quintiles 1 and 2, which included mostly placebo subjects, but decreased (P<0.05) progressively in the three higher quintiles (Table 3 and FIG. 2A). Mean post-treatment iPTH was 166 (10) pg/mL in Quintile 1 and was significantly lower (p<0.001) in Quintiles 3-5, reaching 115 (6), 101 (5) and 97 (5) pg/mL, respectively (FIG. 2B). The observed reductions in iPTH appeared to attenuate as mean serum total 25-hydroxyvitamin D approached the highest level. The proportion of subjects who attained a mean decrease of ≥30% in plasma iPTH from pre-treatment baseline in the EAP was 8.5% in Quintiles 1 and 2, and then increased in a linear fashion to 27.8% in Quintile 3, 42.3% in Quintile 4 and 57.7% in Quintile 5 (FIG. 3).

Changes in mean (SE) serum CTx-1, P1NP, BSAP and total alkaline phosphatase within a given quintile with treatment duration and across quintiles at the end of treatment were similar to those observed for plasma iPTH (FIGS. 2A and 2B).

Mean post-treatment values were within the laboratory normal ranges for all quintiles except for P1NP, which remained elevated in Quintiles 1-3.

Mean (SE) post-treatment levels of serum calcium and phosphorus were 9.3 (0.05) and 3.8 (0.06) mg/dL, respectively, in Quintile 1 and trended slightly upward across the other four quintiles, reaching 9.45 (0.03) and 4.0 (0.07) mg/dL, respectively, in Quintile 5. Mean (SE) post-treatment values for eGFR and the urine Ca:Cr ratio varied without apparent trends amongst the five quintiles between 27.8 (1.1) and 32.3 (1.6) mL/min/1.73 m², and 0.03 (0.004) to 0.04 (0.006), respectively. Mean (SE) post-treatment levels of serum intact FGF23 in Quintiles 1 to 5 were 51.7 (9.6), 63.3 (16.1), 50.6 (8.7), 44.9 (7.5) and 62.8 (7.9) pg/mL, respectively. No significant differences were observed between Quintile 1 and any of the higher quintiles (P=NS) for these five parameters.

SHPT progression, defined as an increase in EOT iPTH>10% from pre-treatment baseline, was also calculated for each quintile.

Mean (SE) plasma iPTH levels at baseline and at EOT are summarized in Table 4 below by post-treatment serum total 25-hydroxyvitamin D quintile, along with the percentage of subjects experiencing SHPT progression.

TABLE 2

| Serum 25D | | | | | |
|---|---|---|---|---|---|
| Quintile | | | Plasma iPTH | | |
| | EOT | | Baseline | EOT | Subjects |
| | ng/mL | | pg/mL | pg/mL | with SHPT |
| | mean | n | mean (SE) | mean (SE) | Progression % |
| 1 | 13.9 | 71 | 157 (8) | 166 (10) | 36.6 |
| 2 | 26.2 | 71 | 141 (6) | 149 (10) | 33.8 |
| 3 | 50.8 | 72 | 142 (7) | 115 (6)^{a} | 9.7^{a} |
| 4 | 68.9 | 71 | 134 (4) | 101 (5)^{a} | 2.8^{a} |
| 5 | 92.5 | 71 | 147 (7) | 97 (5)^{a} | 4.2^{a} |

^{a}Reduced from Quintle 1, p < 0.05

More than one-third of subjects receiving inadequate vitamin D replacement therapy (Quintiles 1 and 2) experienced SHPT progression. Mean iPTH and the percentage of subjects with SHPT progression were reduced only when mean serum total 25D was increased with ERC treatment to at least 51 ng/mL. The results show that attenuation of SHPT progression in Stage 3-4 CKD requires a serum total 25-hydroxyvitamin D target above 50 ng/mL.

This post-hoc analysis of pooled data from two identical 26-week prospective, multicenter randomized, double-blind, placebo-controlled studies conducted with ERC in patients with stage 3 or 4 CKD showed that mean reductions in plasma iPTH and serum bone turnover markers were proportional to increases in mean serum total 25-hydroxyvitamin D and independent of CKD stage. These findings support the conclusion that ERC suppresses elevated iPTH and bone turnover markers by gradually raising the circulating level of 25-hydroxyvitamin D. They further show that reducing iPTH, attenuating SHPT progression, and reducing bone turnover markers in CKD patients requires mean serum 25-hydroxyvitamin D levels of at least 50.8 ng/mL, well above the targets in clinical practice guidelines of 20 or 30 ng/mL, and suggest that normalization of iPTH, if desired, requires even higher levels than those evaluated here. Higher levels of serum 25-hydroxyvitamin D are readily achieved with ERC treatment and proportional to the administered dose. iPTH normalization, however, may not be achievable in view of the apparent attenuation in mean iPTH reduction at the highest level of mean serum total 25-hydroxyvitamin D (92.5 ng/mL) examined herein. This attenuation may be overcome with longer treatment or it may offer both protection from iPTH oversuppression and an indication of the appropriate target for iPTH reduction in patients with stage 3-4 CKD.

The present studies show that gradual elevation of mean serum total 25-hydroxyvitamin D with ERC to levels as high as 92.5 ng/mL over a 26-week period had no adverse effects on mean serum calcium, phosphorus, FGF23, eGFR, VMR or the urine Ca:Cr ratio, and did not increase mean serum 1,25-dihydroxyvitamin D above the ULN (62 pg/mL). Extension of these studies to 52 weeks of ERC treatment demonstrated no increased risks related to these parameters. One observational study has suggested a J-shaped association between serum 25-hydroxyvitamin D levels and all-cause mortality, while another study has shown an increased hazard ratio only at low levels of serum 25-hydroxyvitamin D. In the present studies, a positive correlation was observed between serum total 25-hydroxyvitamin D and 1,25-dihydroxyvitamin D, but no correlation was observed between serum total 25-hydroxyvitamin D and serum calcium or phosphorus. The few episodes of hypercalcemia, observed in 2% of subjects treated with ERC, appeared unrelated to serum total 25-hydroxyvitamin D. Data from the present studies also showed that increasing 25-hydroxyvitamin D exposures not only attenuated the progressive rise in serum bone turnover markers, but actually reduced the levels of these markers, suggesting improved control of high turnover bone disease and a reduction in the risk of related adverse sequelae. Bone degradation and resulting fractures are a significant source of morbidity and mortality in CKD patients with SHPT. Even mildly elevated PTH has recently been demonstrated to produce significant changes in bone architecture and reduce BMD at the spine. Poor bone health has been strongly associated with vascular calcification and the associated high rates of cardiovascular morbidity and mortality in CKD fostering considerable interest in improving bone health and reducing healthcare costs by diagnosing and correcting bone disease in patients with kidney disease.

Surprisingly, ERC treatment had similar effects in patients with either stage 3 or 4 CKD on serum total 25-hydroxyvitamin D and 1,25-dihydroxyvitamin D, and on plasma iPTH. This finding goes against conventional wisdom that calcifediol is less likely to be converted to 1,25-dihydroxyvitamin D₃ as CKD advances, due to declining expression of CYP27B1 in the residual kidneys. However, calcifediol can be activated extra-renally by CYP27B1 in parathyroid and many other tissues. Extra-renal hormone production depends on sufficient circulating levels of 25-hydroxyvitamin D and may be enabled by levels well above 20-30 ng/mL. The present findings indicate that (a) there is adequate renal CYP27B1 activity in predialysis patients to activate 25-hydroxyvitamin D and/or that (b) 25-hydroxyvitamin D is activated by CYP27B1 expressed outside the kidneys and released into circulation. While serum 24,25-dihydroxyvitamin D₃ levels increased with ERC treatment, the VMR rose only moderately, suggesting that there was no substantial induction of CYP24A1.

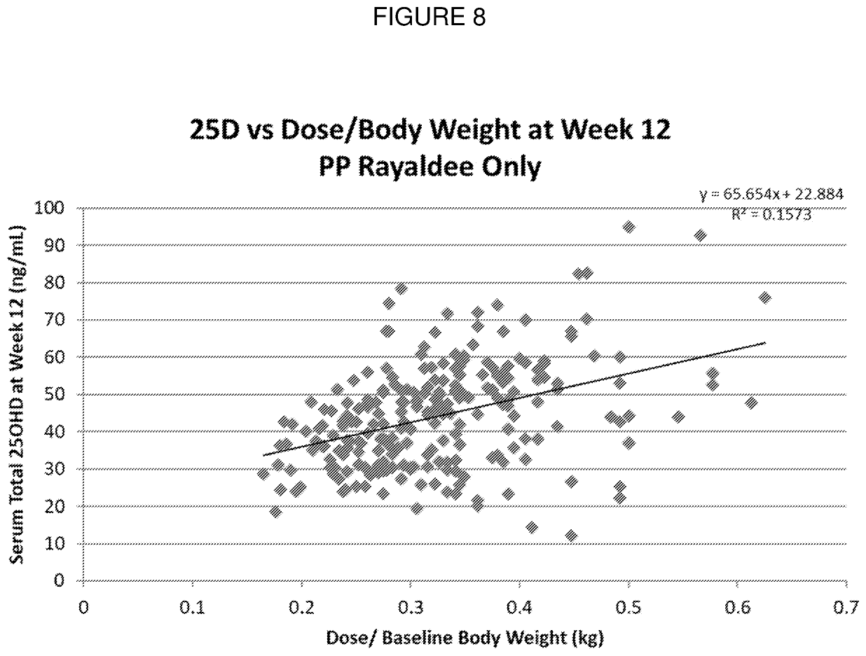

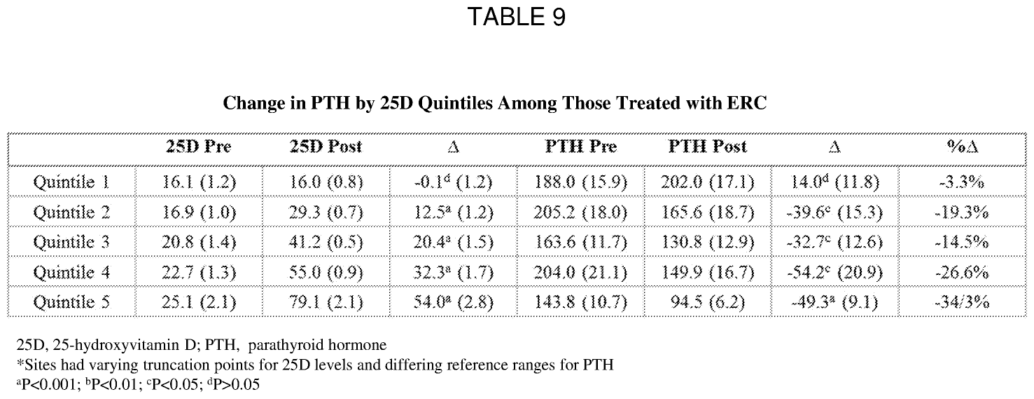

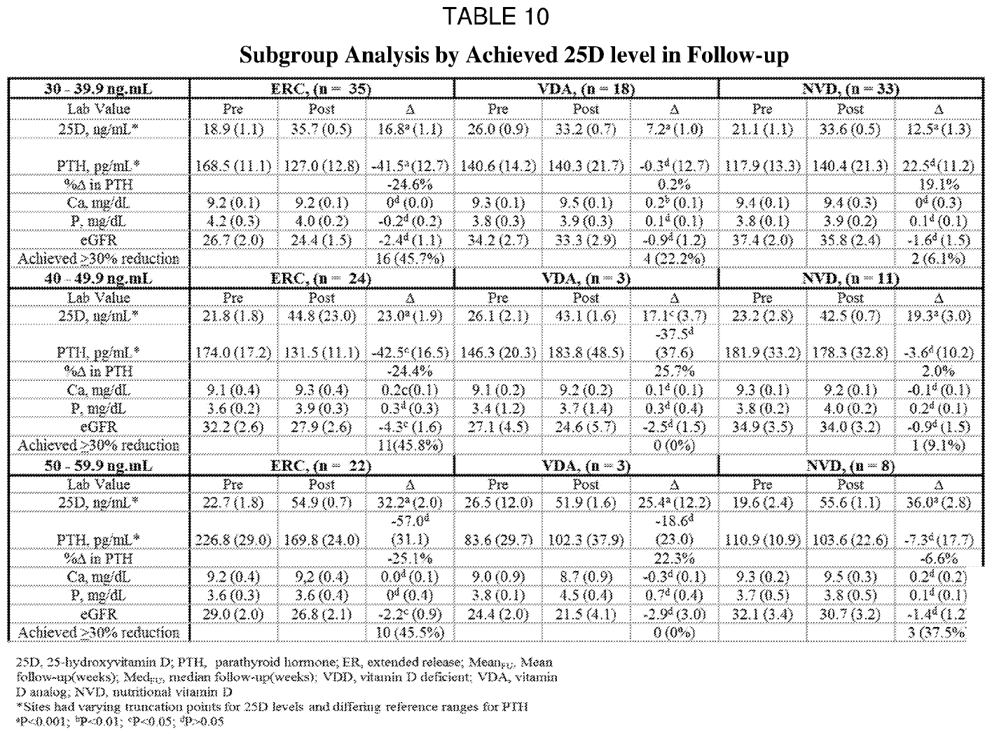

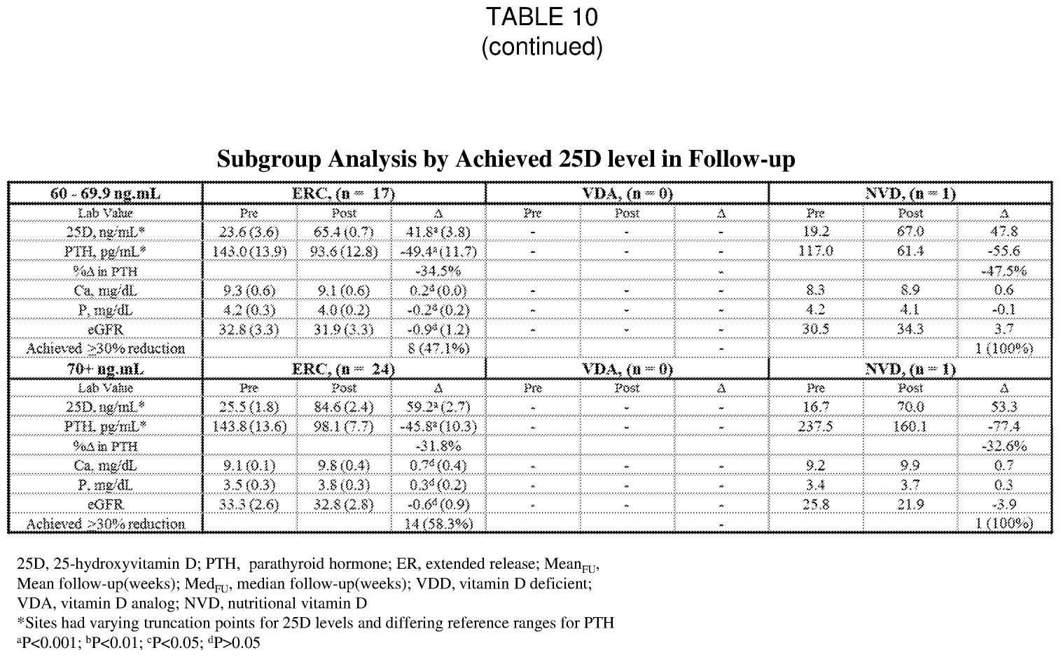

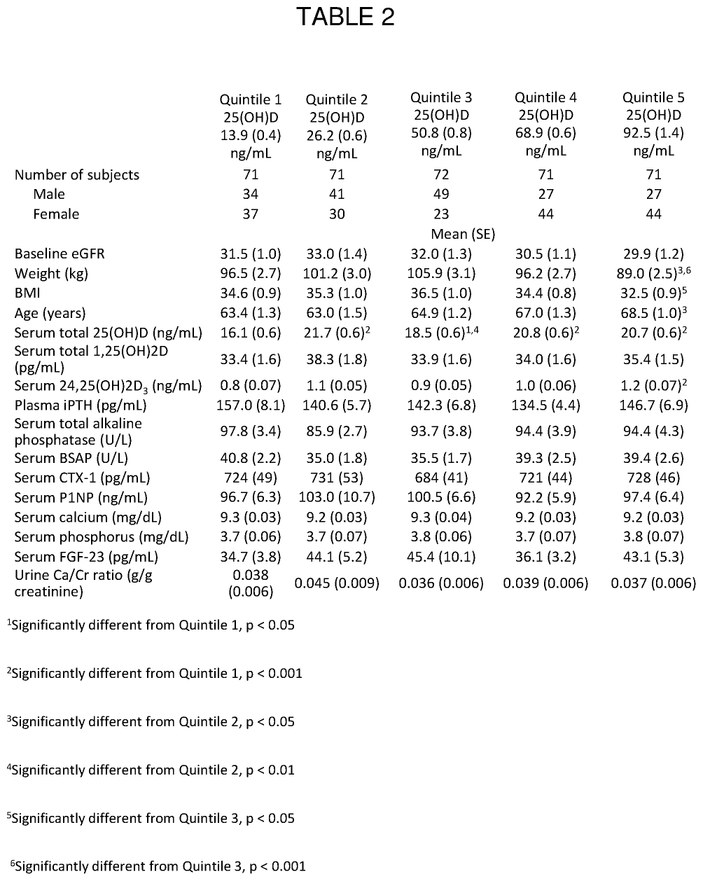

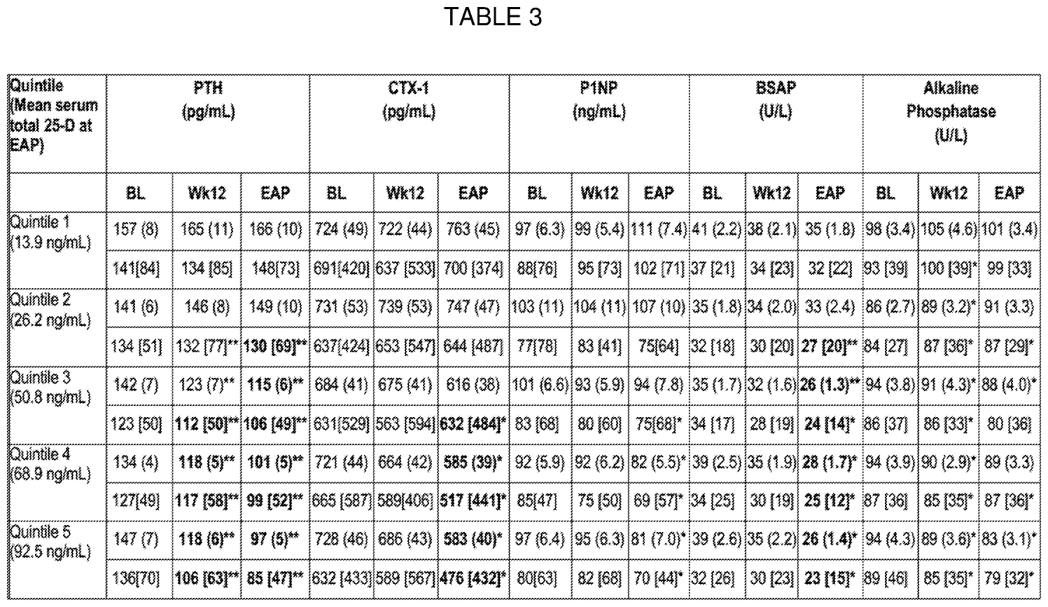

FIGS. 5-8 show the relationship between patient weight and dose response in serum 25-hydroxyvitamin D levels following 12 weeks of treatment with 30 mcg daily ERC. FIGS. 5-6 show the relationship between patient weight at initiation of treatment (baseline) and resulting serum 25-hydroxyvitamin D concentrations (both baseline subtracted serum concentrations, and actual serum concentrations, respectively) after 12 weeks of treatment with 30 mcg daily ERC. FIGS. 7-8 show the relationship between serum 25-hydroxyvitamin D after 12 weeks of treatment with 30 mcg daily ERC and dose per baseline body weight (both baseline subtracted serum concentrations, and actual serum concentrations, respectively).

In conclusion, pooled data from two large prospective RCTs demonstrated that ERC safely increased 25-hydroxyvitamin D exposures in patients with stage 3 or 4 CKD to levels well above those recommended in current clinical practice guidelines. Mean levels of serum total 25-hydroxyvitamin D of at least 50.8 ng/mL were associated with proportional increases in serum 1,25-hydroxyvitamin D, decreases in plasma iPTH and serum bone turnover markers, attenuating SHPT progression defined as increase in EOT iPTH>10% from pre-treatment baseline, and not associated with adverse changes in mean serum calcium, phosphorus, FGF23, eGFR or the urine Ca:Cr ratio. Elevation of mean serum total 25-hydroxyvitamin D to 92.5 ng/mL was insufficient to normalize plasma iPTH, suggesting that higher exposures may be needed to optimally treat SHPT in stage 3 or 4 CKD.

### Example 2

This example describes a structured chart review of patients with Stage 3 or 4 Chronic Kidney Disease who have Vitamin D Insufficiency and Secondary Hyperparathyroidism, and were being treated with Extended-Release Calcifediol or other relevant comparators. This study relates to Mineral and Bone Disorder in Pre-dialysis: A Real-World Assessment of Risk and Effectiveness of Current SHPT Treatment Approaches (MBD-AWARE).

Objectives

The overall objectives of this study were to generate preliminary real-world evidence demonstrating: (a) the safety and effectiveness of extended-release calcifediol (ERC) for treating secondary hyperparathyroidism (SHPT) in adult patients with stage 3 or 4 chronic kidney disease (CKD) and vitamin D insufficiency (VDI): and, (b) the utilization, safety, and effectiveness of other vitamin D therapies (OVDT) considered to be standard of care for SHPT in treating these patients. OVDT consist of nutritional vitamin D (NVD), defined as orally administered ergocalciferol or cholecalciferol, or active (1α-hydroxylated) vitamin D analogs (VDA), defined as orally administered calcitriol, paricalcitol, or doxercalciferol.

The specific objectives of the study were to describe or estimate the following in each of three cohorts (defined below in Study Design) before and during a follow-up period of six months: (1) changes in serum calcium and phosphorus; (2) changes in serum total 25-hydroxyvitamin D (25D) and parathyroid hormone (PTH) levels; (3) achievement of normal 25D levels; (4) achievement of ≥30% PTH reduction; and (5) changes in ancillary laboratory values.

### Background

ERC 30 mcg capsules were approved by the Food and Drug Administration in June 2016 as a treatment for SHPT in adult patients with stage 3 or 4 CKD and VDI. The active ingredient, calcifediol, is 25-hydroxyvitamin D₃, the physiological precursor to and VDI the vitamin D hormone, 1,25-dihydroxyvitamin D₃ (calcitriol). Calcifediol is synthesized by the liver from vitamin D₃ (cholecalciferol), which is generated endogenously in skin following exposure to sunlight or obtained from the diet or supplements. Another prohormone, 25-hydroxyvitamin D₂, is synthesized hepatically from vitamin D₂ (ergocalciferol), which cannot be produced endogenously and is obtained only from the diet or supplements. These two prohormones are collectively referred to as “25-hydroxyvitamin D.” Unless an individual is receiving significant ergocalciferol supplementation, essentially all of the 25-hydroxyvitamin D in blood consists of calcifediol.

CKD is a steadily increasing health problem in the United States (US) driven by an aging population and an increasing prevalence of obesity with associated complications of hypertension and diabetes mellitus. CKD is categorized into five stages, each defined by an estimated glomerular filtration rate (eGFR) range that progressively decreases from stage 1 to 5. Aberrations in mineral metabolism and bone histology begin early in the course of CKD, worsening as, eGFR declines [Levin et al 2007]. Even minimal reductions in, eGFR have been linked to increased risk of bone loss (osteoporosis) and incidence of hip fracture. Co-morbidities associated with CKD include SHPT, VDI, pervasive soft tissue calcification, cardiovascular (CV) disease, infections and reduced quality of life [Souberbielle et al 2010].

Vitamin D Insufficiency (VDI) in patients with CKD is driven by nutritional inadequacy, decreased exposure to sunlight, proteinuria, decreased hepatic synthesis of calcifediol and excessive expression of the vitamin D catabolic enzyme, CYP24A1 [Helvig et al 2010]. It is widely accepted that serum total 25D is the best indicator of a patient's vitamin D status. Serum total 25-hydroxyvitamin D (25D) levels of ≥30 ng/mL are considered adequate in CKD patients while levels of <30 ng/mL are considered “insufficient” [Holick et al 2011]. The commonly used reference range for serum total 25D is 30 to 100 ng/mL [Souberbielle et al 2010]. Observational studies suggest that in CKD patients, as glomerular filtration rate (GFR) declines, higher 25D levels may be required to achieve PTH targets [Ennis et al 2016].

Levels of serum total 25D in the general population vary according to many factors, including intensity of sunlight (varying with geographic location and season), exposure to sunlight (affected by skin pigmentation, use of sunscreen and other cultural factors), age and dietary intake [Holick 1995]. Levels tend to be lower during the winter and at higher latitudes. In patients with CKD, low total serum 25D levels (VDI) are unrelated to season or latitude and become more prevalent as kidney disease advances.

Because renal and extra-renal production of calcitriol is dependent on an adequate supply of calcifediol, VDI causes inadequate calcitriol production. Declining renal function further impairs the conversion of calcifediol to calcitriol by the renal 1α-hydroxylase (CYP27B1). Chronically low circulating calcitriol results in decreased intestinal absorption of dietary calcium, increased secretion of PTH by the parathyroid glands and, ultimately, SHPT.

Clinical practice guidelines for the treatment of SHPT in CKD recommend regular screening for elevated PTH beginning in patients with stage 3 CKD. The guidelines issued by the National Kidney Foundation from the Kidney Disease Outcomes Quality Initiative (KDOQI) [National Kidney Foundation. KDOQI Clinical Practice Guidelines for Bone Metabolism and Disease in Chronic Kidney Disease 2003, Guideline 8A], the more recent Kidney Disease Improving Global Outcomes (KDIGO) Clinical Practice Guideline for the Diagnosis, Evaluation, Prevention, and Treatment of Chronic Kidney Disease-Mineral and Bone Disorder (CKD-MBD) [Kidney Disease: Improving Global Outcomes (KDIGO) CKD-MBD Work Group 2009] and the recent update to the KDIGO guideline [KDIGO 2017 Clinical Practice Guideline Update for the Diagnosis, Evaluation, Prevention, and Treatment of CKD-MBD], also recommend testing for VDI when elevated PTH is encountered, and correcting with aggressive vitamin D supplementation. However, the medical literature documents that in patients with stage 3 or 4 CKD low serum total 25D is inconsistently or inadequately treated by NVD (ergocalciferol or cholecalciferol) supplementation, and elevated PTH remains uncorrected by NVD. More than 30 studies have been published since 1973 in which ergocalciferol or cholecalciferol was administered to patients with stage 3 to 5 CKD. The overall conclusion from this body of work is summarized by Kalantar-Zadeh and Kovesdy: “Most of these studies have shown either no or minimal to inadequate changes in PTH levels, usually only in some stages of CKD, or changes that still would not satisfy the K/DOQI recommended target ranges for PTH”[Kalantar-Zadeh and Kovesdy 2009]. A more recent review of the published randomized clinical trials concluded that vitamin D had no efficacy in lowering iPTH levels in patients with Stage 3 to 5 CKD [Agarwal and Georgianos 2016]. Hence, there is a need for effective treatment to increase serum total 25D and control elevated iPTH in this patient population. ERC is intended to meet this pressing need.

Rationale

Evidence of the safety and efficacy of ERC as a treatment for SHPT in adult patients with stage 3 or 4 CKD and VDI has been demonstrated in randomized, controlled clinical trials [Sprague et al 2016, Sprague et al 2017]. However, given the recency of marketing authorization, data originating from real-world settings is lacking on the utilization, safety, and effectiveness of ERC in these patients. In addition, there is limited evidence on the current utilization, safety, and effectiveness of OVDT which are considered to be the standard of care. This retrospective study was intended to generate new real-world evidence on the utilization, safety, and effectiveness of ERC and OVDT in the US.

Study Design and Methodology

Eighteen US nephrology clinics took part in the study and provided medical records of the first 376 patients who met the study inclusion criteria for retrospective analysis. A screening tool was developed and served as a data entry portal for study participating clinics. Data was collected until the cut-off date as decided by study researchers.

Study Population

Three hundred and seventy-six patients were recruited and entered for this retrospective study. Patients were considered to have met the study inclusion criteria if documented as having stage 3 or stage 4 CKD, a history of VDI and SHPT, and initiated treatment with ERC, NVD or a VDA on or after a date. 1,917 subjects were known to be screened for eligibility. However, the true number is not known, as sites may have screened subjects before attempting or entering eligible patients in the data collection tool. Those who met inclusion criteria were grouped into the following three cohorts: 1) ERC Any Use (ERCAU) Cohort, defined as patients who had used ERC for minimum of 1 month; 2) Nutritional Vitamin D (NVD) Cohort, defined as patients who had used nutritional vitamin D (ergocalciferol or cholecalciferol) for minimum of 1 month; and, (nutritional vitamin D was converted to and displayed as total weekly dose); and, 3) Active Vitamin D Analog (VDA) Cohort, defined as patients who have used an active (1α-hydroxylated) vitamin D analog (calcitriol, paricalcitol or doxercalciferol) for a minimum of 1 month.

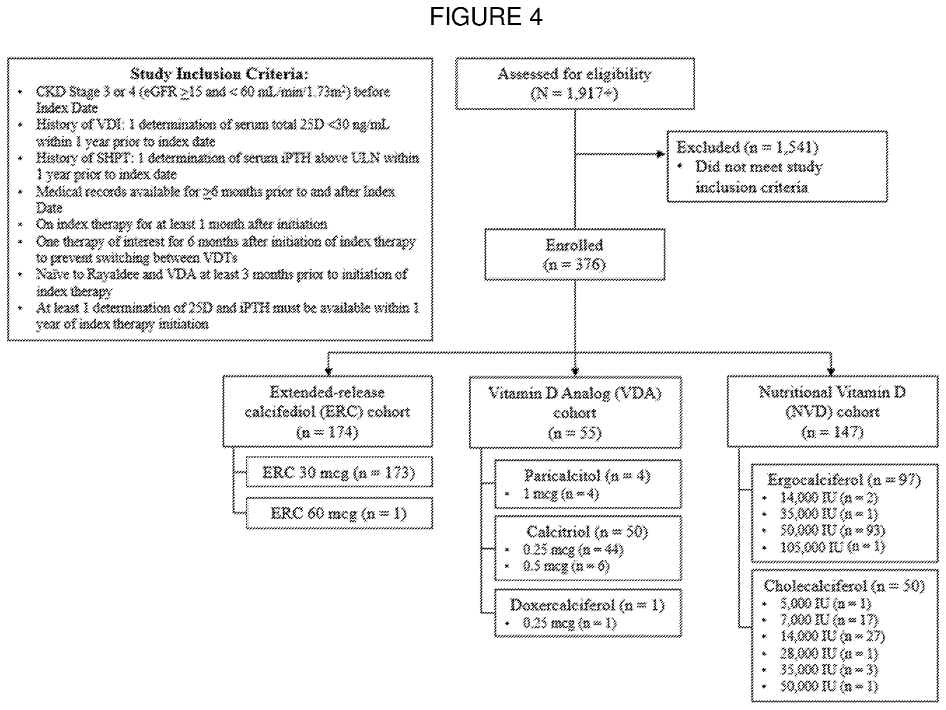

FIG. 4 shows a patient distribution between study cohorts.

Inclusion Criteria

All patients included in the study required to have a history of SHPT (PTH above the laboratory upper limit of normal (ULN)), VDI (serum total 25D below 30 ng/mL), and stage 3 or 4 CKD (eGFR of ≥15 to <60 mL/min/1.73 m²) before the Index Date (defined below) and meet the criteria for inclusion in one of the three cohorts listed above (see Study Population).

All included patients were assigned an Index Date, defined as the date of the first treatment with ERC or OVDT. Upon defining the Index Date, the following criteria was required to be met for each patient for them to be considered eligible for this study:
Medical records available for months prior to the Index Date (baseline records);
Medical records available for months after the Index Date (follow-up records);
Only taking one SHPT therapy of interest during the follow-up period and not switching therapies during this time, with the exception that changes in active (1α-hydroxylated) VDA;
Patients naïve to Rayaldee and VDA during the three months prior to the index date;
At least one serum total 25D and PTH determination available within one year prior to the index date; and,
At least one 25D and PTH determination available at or after six months beyond the Index Date and within 30 days of treatment cessation.

Source Data

The source data for this study included the following:
Electronic or Paper Medical Records: Data on diagnoses, past medical history, treatments, and other resource utilization were captured for six months prior to the Index Date and for at least 6 months after the Index Date. The data type, level, and date of collection was collected. It is possible that patients may have sought care outside of the selected facilities, particularly outpatient visits related to comorbid conditions associated with CKD, such as CV disease, so some medical records may have been incomplete (as it is not always possible to capture resource utilization from other facilities). Data points where potential information bias may have been introduced were captured across all sites and flagged. Other data points that were available for some, but not all sites, and for some, but not all patients within a clinic, were also flagged and analyzed in subgroup analyses. Other medical records from other sites of care, when available, including emergency room visits, outpatient visits, and pharmacy information were incorporated to supplement patient data.
Laboratory Databases: Any clinical laboratory data of interest, including 25D, PTH, fibroblast growth factor 23 (FGF23), calcium, phosphorus, hemoglobin, eGFR/creatinine, urinary protein, cholesterol, albumin, C-reactive protein, fibrinogen, homocysteine, and calcium phosphorous product, were captured for six months prior to the Index Date and for 6 months after the Index Date. The lab type, level, and date of collection were captured. The 25D and PTH levels were collected for up to one year prior and after the Index Date.

Results

Patients who met study inclusion criteria were included in the study. Three hundred and seventy-six (19.6%) out of 1,917 patients were screened and assessed for study eligibility and enrolled in the study. Of the enrolled patients, 174 (46.3%) initiated treatment with ERC, 55 (14.6%) with VDAs, and 147 (39.1%) with NVD. These patients were assigned to cohorts based on their index criteria (as defined in Section 3.1). FIG. 4 displays the study CONSORT diagram and outlines the numbers for patient and by index dose (converted to and displayed as weekly dose).

Within the ERC cohort, 173 (99.4%) of patients initiated treatment with 30 mcg per day. Calcitriol was the most common vitamin D therapy (90.9%) prescribed within the VDA cohort. In the NVD cohort, ergocalciferol was prescribed to 66.0% of patients with 50,000 IU per month being the most commonly prescribed dose

The enrolled cohort had a mean (SE) age of 69.5 (13.2) years. The cohort was approximately evenly distributed between men (49.2%) and women (50.8%) and predominantly Non-Hispanic (88.8%) and Caucasian (64.6%). Subjects had a mean (SD) height of 167.6 (12.0) cm, weight of 90.8 (25.0) kg, and body mass index (BMI) of 32.8 (15.2). The primary cause of CKD was only listed in 113 (30.2%) subjects, with hypertension (55.6%) and diabetes (38.9%) being the most common among known causes. An eGFR was calculated at baseline using the Modified Diet for Renal Disease (MDRD) equation. There were more enrolled patients with CKD Stage 3 (54.3%) compared to CKD Stage 4 (45.7%). The three most common comorbidities were diabetes (51.6%), hypertension (80.6%), and anemia (40.2%). A total of 71 (18.9%) of patients took anemia medications concomitantly while only 14 (3.7%) received phosphate binders.

Age, gender, race, and height were similar across the three index therapy cohorts. The ERC cohort was comprised of nearly twice the proportion of Hispanics (15.5%) as compared to the VDA (7.3%) and NVD (7.5%) cohorts. On average, those treated with ERC had a higher BMI (34.2) than those treated with VDA (29.4) and NVD (32.4). Additionally, the primary cause of CKD was similar across cohorts. The majority of patients treated with ERC and VDA were CKD Stage 4, 53.4% and 61.8%, respectively, while most patients treated with NVD were CKD Stage 3 (69.4%). Furthermore, there were varying rates of comorbidities across cohorts. Despite many similarities across cohorts, there existed subtle differences among treatment groups which may have contributed to variations in treatment efficacy. Further details can be found in Table 5.

TABLE 5

| | Full | ERC, | VDA, | NVD, |
|---|---|---|---|---|
| Variable | (n = 374) | (n = 174) | (n = 55) | (n = 147) |
| Age, Mean | 69.5 (13.2) | 69.0 (13.2) | 71.8 (13.1) | 69.3 (13.4) |
| (SD) | | | | |
| Male, n (%) | 185 (49.2%) | 84 (48.3%) | 30 (54.5%) | 71 (48.3%) |
| Hispanic, | 42 (11.2%) | 27 (15.5%) | 4 (7.3%) | 11 (7.5%) |
| n (%) | | | | |
| Race, n (%) | | | | |
| Caucasian | 243 (64.6%) | 113 (64.9%) | 35 (63.6%) | 95 (64.6%) |
| African | 75 (19.9%) | 34 (19.5%) | 10 (18.2%) | 31 (21.1%) |
| American | | | | |
| Asian | 1 (0.3%) | 0 (0%) | 0 (0%) | 1 (0.7%) |
| American | | | | |
| Native | 1 (0.3%) | 0 (0%) | 1 (1.8%) | 0 (0%) |
| American | | | | |
| Other | 27 (7.2%) | 19 (10.9%) | 2 (3.6%) | 6 (4.1%) |
| Not | 29 (7.7%) | 8 (4.6%) | 7 (12.7%) | 14 (9.5%) |
| Available | | | | |
| Height | 167.6 (12.0) | 167.1 (13.7) | 168.0 (9.0) | 167.9 (10.8) |
| in cm, | | | | |
| Mean (SD) | | | | |
| Weight | 90.8 (25.0) | 92.4 (25.9) | 83.3 (22.3) | 91.6 (13.4) |
| in kg, | | | | |
| Mean (SD) | | | | |
| BMI, Mean | 32.8 (15.2) | 34.2 (20.7) | 29.4 (7.2) | 32.4 (7.6) |
| (SD) | | | | |
| Primary | | | | |
| Cause of | | | | |
| CKD, n (%) | | | | |
| Known | n = 113 | n = 69 | n = 13 | n = 31 |
| cause | | | | |
| Diabetes | 44 (38.9%) | 30 (43.5%) | 2 (15.4%) | 12 (38.7%) |
| Hyper- | 64 (55.6%) | 36 (52.2%) | 11 (84.6%) | 17 (54.8%) |
| tension | | | | |
| Other | 5 (4.4%) | 3 (4.3%) | 0 (0.0%) | 2 (6.5%) |
| CKD Stage, | | | | |
| n (%) | | | | |
| CKD | 204 (54.3%) | 81(46.6%) | 21 (38.2%) | 102 (69.4%) |
| Stage 3 | | | | |
| CKD | 172 (45.7%) | 93 (53.4%) | 34 (61.8%) | 45 (30.6%) |
| Stage 4 | | | | |
| Co- | | | | |
| morbidities, | | | | |
| n (%) | | | | |
| Diabetes | 194 (51.6%) | 90 (51.7%) | 29 (52.7%) | 75 (51.0%) |
| Hyper- | 303 (80.6%) | 128 (73.6%) | 46 (83.6%) | 129 (87.8%) |
| tension | | | | |
| Anemia | 151 (40.2%) | 67 (38.5%) | 23 (41.8%) | 61 (41.5%) |
| Heart | 48 (12.6%) | 14 (8.0%) | 10 (18.2%) | 24 (16.3%) |
| Failure | | | | |
| Coronary | 42 (11.2%) | 17 (9.8%) | 5 (9.1%) | 20 (13.6%) |
| artery | | | | |
| disease | | | | |
| Angina | 3 (0.8%) | 1 (0.6%) | 1 (1.8%) | 1 (0.7%) |
| Peripheral | 7 (1.9%) | 3 (1.7%) | 1 (1.8%) | 3 (2.0%) |
| vascular | | | | |
| disease | | | | |
| Cerebral | 4 (1.1%) | 3 (1.7%) | 1 (1.8%) | 0 (0.0%) |
| vascular | | | | |
| disease | | | | |
| Cancer | 4 (1.1%) | 3 (1.7%) | 0 (0.0%) | 1 (0.7%) |
| Hyper- | 132 (35.1%) | 48 (27.6%) | 16 (29.1%) | 68 (46.3%) |
| lipidemia | | | | |
| None | 50 (13.3%) | 39 (22.4%) | 4 (7.3%) | 7 (4.8%) |
| Con- | | | | |
| comitant | | | | |
| Medications, | | | | |
| n (%) | | | | |
| Phosphate | 14 (3.7%) | 6 (3.4%) | 3 (5.5%) | 5 (3.4%) |
| Binders | | | | |
| Anemia | 71(18.9%) | 25 (14.4%) | 15 (27.3%) | 31 (21.1%) |
| Medications | | | | |

Primary Analysis

The primary analysis assessed key clinical effectiveness (25D and PTH) and safety (serum Ca and P) laboratory values, as well as eGFR among all enrolled patients before and after index therapy treatment initiation. Results were categorized by index therapy cohort (Table 3).

For the 174 ERC patients, baseline 25D and PTH levels averaged 20.3±0.7 (SE) ng/mL and 181±7.4 pg/mL, respectively. ERC treatment raised 25D by 23.7±1.6 ng/mL (p<0.001) and decreased PTH by 34.1±6.6 pg/mL (p<0.001) without statistically significant on serum calcium and phosphorus levels. Additionally, eGFR decreased 3.1±0.7 mL/min/1.73 m² (p<0.001).

For the 55 VDA patients, baseline 25D and PTH levels averaged 23.5±1.0 (SE) ng/mL and 156.9±9.7 pg/mL, respectively. VDA treatment raised 25D by 5.5±1.3 ng/mL (p<0.001) without statistically significant impact on PTH and serum phosphorus levels. Additionally, serum calcium levels elevated 0.2±0.1 mg/dL (p<0.001) and eGFR decreased 1.6±0.6 (p<0.01).

For the 147 NVD patients, baseline 25D and PTH levels averaged 18.8±0.6 (SE) ng/mL and 134.8±6.8 pg/mL, respectively. NVD treatment raised 25D by 9.7±1.5 ng/mL (p<0.001) without statistically significant impact on PTH, serum calcium and phosphorus levels. Additionally, eGFR decreased 1.2±0.6 (p<0.05). The average weekly dose for NVD patients was 38,392.2 IU.

On average, patients treated with ERC saw the greatest 25D increase during follow-up. Additionally, ERC was the only treatment that resulted in a significant mean decrease in PTH levels and only patients treated with ERC saw a mean increase to normal levels of 25D (≥30 ng/mL). Other than an increase in serum Ca among patients treated with VDA, there was no impact of any treatment on calcium or phosphorus levels (Table 6).

Additional analyses assessing effectiveness per the clinical trial endpoints (NCT01651000) were conducted. Among the 174 patients treated with ERC, 122 (70.1%) achieved serum 25D≥30 ng/mL and 71 (40.8%) achieved a ≥30% reduction in PTH levels. Among the 55 patients treated with VDA, 24 (43.6%) achieved 25D≥30 ng/mL and 12 (21.8%) achieved a ≥30% reduction in PTH levels. Among the 147 patients treated with NVD, 54 (36.7%) achieved 25D≥30 ng/mL and 22 (15.0%) achieved a ≥30% reduction in PTH levels. Across cohorts, patients treated with ERC had the highest percentage of achieving clinical trial endpoints vs VDA and NVD. Additionally, patients treated with ERC had the lowest proportion of patients with a PTH increase of 10% or more. Additional analyses were also conducted on whether a patient had 25D<20 ng/mL at baseline and whether they achieved PTH<70 pg/mL at follow-up (Table 7).

Additionally, an analysis of SHPT progression was carried out. SHPT progression was defined as an increase in PTH of at least 10% from baseline. Table 8 provides the results of each cohort.

TABLE 8

| | ERC | VDA | NVD |
|---|---|---|---|
| Total # patients in cohort | 174 | 55 | 147 |
| # patients experiencing SHPT | 38 | 17 | 58 |
| progression (% of total in cohort) | (21.8%) | (30.9%) | (39.5%) |

As shown in Table 8, the cohort of patients treated with ERC had the lowest percentage of patients experiencing SHPT progression compared to the other two cohorts.

Secondary Analysis

Percent change in iPTH Analysis—25D Quintiles Among those Treated with ERC

A secondary analysis was performed to assess change in PTH among those treated with ERC. This analysis split subjects into five equal groups based on 25D level in the post treatment initiation laboratory. Among those in Quintile 1, there was no statistically significant impact on PTH. Quintiles 2, 3, and 4 achieved an average PTH reduction of 19.3%, 14.5%, and 26.6%, respectively. A mean PTH reduction of >30% was not achieved until Quintile 5, where 25D was increased to a mean of 79.1±(2.1) ng/mL (Table 9).

An additional secondary analysis was performed to assess change in PTH among those across cohorts. Additionally, safety (serum Ca and P) laboratory measures as well as the clinical endpoint of >30% PTH reduction. Results were broken up by index therapy cohort. This analysis split subjects into groups based on index therapy and level of 25D achievement. A mean PTH reduction of >30% was not achieved until 25D was increased to >60 ng/mL. While 0 (0%) patients treated with VDA and only 2 (1.3%) patients treated with NVD achieved 25D>60 ng/mL, it was achieved in 41 (23.6%) patients treated with ERC. Across all 25D achievement levels, treatment with ERC had no impact on serum calcium or phosphate (Table 10).

Key takeaways from this study include the following:
On average, ERC was the only treatment that raised 25D to normal levels (≥30 ng/mL).
In nearly all subgroup analyses, treatment with ERC had no impact on key safety markers (serum calcium and phosphorus).
Subjects treated with ERC in the real-world saw the highest rate of achieving ≥30 ng/mL and ≥30% reduction in iPTH in the follow-up period.
When assessing iPTH reduction by level of 25D achievement, a mean iPTH reduction of ≥30% was not achieved until 25D was increased to ≥60 ng/mL.
Despite differences in patient demographics and clinical characteristics at baseline, patients in the real-world saw similar clinical effectiveness and safety outcomes when compared to the clinical trial results.
However, differences in total change of 25D in ERC cohort may be due to dose titration or concomitant medication use.
When assessing key clinical trial endpoints treatment with ERC in the real-world similar results are obtained as compared to the clinical trial.
122 (70.1%) patients treated with ERC achieved >30 ng/mL in at follow-up compared to 80%-83% in the clinical trial.
71 (40.8%) patients treated with ERC achieved >30% reduction in PTH at follow-up compared to 33%-34%.
The variation in baseline levels, dosing patterns, and concomitant medications may result in differences of achieving clinical trial endpoints.
Adherence to dose titration recommendation of 60 mcg/day may lead to further increases to 25D and reductions to PTH levels.

### CONCLUSIONS

This chart review demonstrates that treatment with ERC in the real-world resulted in similar clinical effectiveness and safety outcomes as the clinical trial, despite a more severe population and lower levels of dosing. Additionally, similar achievement rates of clinical trial endpoints were produced in a real-world setting. While the real-world saw low rates of dose titrations, increasing ERC dose may lead to further increases of 25D and reductions of PTH levels in the real-world. Overall, this study provides strong evidence that the clinical effectiveness and safety of ERC treatment is maintained in the real-world setting.

### REFERENCES

Agarwal R, Georgianos P I. Con: Nutritional vitamin D replacement in chronic kidney disease and end-stage renal disease. Nephrol Dial Transplant 2016 31:706-713.
Ennis J L, Worcester E M, Coe F L et al. Current recommended 25-hydroxyvitamin D targets for chronic kidney disease management may be too low. J Nephrol. 2016 29: 63-70.
Helvig C F, Cuerrier D, Hosfield C M et al. Dysregulation of renal vitamin D metabolism in the uremic rat. Kidney Int. 2010 78: 463-472.
Holick M F. Environmental factors that influence the cutaneous production of vitamin D. Am J Clin Nutr. 1995 61: 638S-645S.
Holick M F, Binkley N C, Bischoff-Ferrari H A, et al. Evaluation, treatment, and prevention of vitamin D deficiency: an Endocrine Society clinical practice guideline. J Clin Endocrinol Metab. 2011; 96:1911-1930.
Kalantar-Zadeh K, Kovesdy C P. Clinical outcomes with active versus nutritional vitamin D compounds in chronic kidney disease. Clin J Am Soc Nephrol. 2009 4: 1529-1539.
Kidney Disease: Improving Global Outcomes (KDIGO) CKD-MBD Work Group. KDIGO clinical practice guideline for diagnosis, evaluation, prevention, and treatment of chronic kidney disease-mineral and bone disorder (CKD-MBD). Kidney Int. 2009 76: S1-5130
Kidney Disease Improving Global Outcomes (KDIGO) 2017 clinical practice guideline update for the diagnosis, evaluation, prevention and treatment of chronic kidney disease—mineral and bone disorder (CKD-MBD). Kidney Int Suppl. 2017;7:S1-S59.
Levin A, Bakris G L, Molitch M et al. Prevalence of abnormal serum vitamin D, PTH, calcium, and phosphorus in patients with chronic kidney disease: results of the study to evaluate early kidney disease. Kidney Int. 2007 71:31-38.
National Kidney Foundation. K/DOQI Clinical Practice Guidelines for Bone Metabolism and Disease in Chronic Kidney Disease. Am J Kidney Dis. 2003 42: S1-S202.
Souberbielle J, Body J, Lappe J M et al. Vitamin D and musculoskeletal health, cardiovascular disease, autoimmunity and cancer: Recommendations for clinical practice. Autoimmun Rev. 2010 9: 709-715.
Sprague, S M et al. Use of Extended-Release Calcifediol to Treat Secondary Hyperparathyroidism in Stages 3 and 4 Chronic Kidney Disease. Am J Nephrol 2016 44(4) 316-325.
Sprague, S M et al. Extended-release calcifediol for secondary hyperparathyroidism in stage 3-4 chronic kidney disease. Expert Rev Endocrinol Metab. 2017; 12(5):289-301.

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

1.-81. (canceled)

82. A method of preventing, halting, or reversing SHPT progression in a patient, defined as an increase in plasma iPTH>10% from pre-treatment baseline, comprising effective administration of 25-hydroxyvitamin D to increase and maintain serum total 25-hydroxyvitamin D in the patient to a concentration greater than 50 ng/mL, and thereby preventing, halting, or reversing SHPT progression in the patient.

83. A method of preventing, halting, or reversing SHPT progression in a population of patients, defined as an increase in plasma iPTH>10% from pre-treatment baseline, comprising effective administration of 25-hydroxyvitamin D to increase and maintain serum total 25-hydroxyvitamin D in the patients to a mean concentration greater than 50 ng/mL and thereby preventing, halting, or reversing SHPT progression in the patient population, wherein the fraction of subjects experiencing SHPT progression is less than 10%.

84. A method of treating a disease, condition, or disorder associated with an increase in iPTH from baseline in a patient in need of treatment thereof, comprising effective administration of 25-hydroxyvitamin D to increase and maintain the patient's serum total 25-hydroxyvitamin D in a range of about 50 to about 300 ng/mL during chronic administration, and thereby treat the disease, condition, or disorder.

85. The method of claim 82, comprising increasing 25-hydroxyvitamin D to maintain serum total 25-hydroxyvitamin D level in the patient to a concentration in a range of greater than 50 ng/mL to about 300 ng/mL.

86. The method of claim 85, comprising increasing 25-hydroxyvitamin D to maintain serum total 25-hydroxyvitamin D level in the patient to a concentration in a range of greater than 50 ng/mL to about 200 ng/mL.

87. The method of claim 86, comprising increasing 25-hydroxyvitamin D to maintain serum total 25-hydroxyvitamin D level in the patient to a concentration in a range of greater than 50 ng/mL to about 100 ng/mL.

88. The method of claim 84, comprising increasing 25-hydroxyvitamin D to maintain serum total 25-hydroxyvitamin D level in the patient to a concentration in a range about 50 ng/mL to about 200 ng/mL.

89. The method of claim 88, comprising increasing 25-hydroxyvitamin D to maintain serum total 25-hydroxyvitamin D level in the patient to a concentration in a range of about 50 ng/mL to about 100 ng/mL.

90. The method of claim 82, wherein the effective administration of 25-hydroxyvitamin D comprises avoiding significant increase in the patient's corrected serum calcium level, compared to pre-treatment baseline.

91. The method of claim 82, wherein the effective administration of 25-hydroxyvitamin D comprises avoiding significant increase in the patient's serum phosphorous level, compared to pre-treatment baseline.

92. The method of claim 82, wherein the effective administration of 25-hydroxyvitamin D comprises avoiding significant increase in the patient's serum FGF23 level, compared to pre-treatment baseline.

93. The method of claim 82, wherein the effective administration of 25-hydroxyvitamin D comprises providing an average daily rise in serum total 25-hydroxyvitamin D during increase of serum total 25-hydroxyvitamin D of 3 ng/mL or less.

94. The method of claim 91, wherein the effective administration of 25-hydroxyvitamin D comprises providing an average daily rise in serum total 25-hydroxyvitamin D during increase of serum total 25-hydroxyvitamin D of at least 0.2 ng/mL.

95. The method of claim 82, wherein the effective administration of 25-hydroxyvitamin D comprises increasing serum total 25-hydroxyvitamin D to a steady state level in a range of about 50 to about 300 ng/mL, over a period of at least 8 weeks.

96. The method of claim 82, wherein the effective administration of 25-hydroxyvitamin D comprises increasing the patient's serum total 1,25-dihydroxyvitamin D to a steady state level of at least 40 pg/mL.

97. The method of claim 82, wherein the effective administration of 25-hydroxyvitamin D comprises increasing serum total 1,25-dihydroxyvitamin D to a steady state level of not more than 62 pg/mL.

98. The method of claim 82, wherein the patient is vitamin D insufficient at initiation of therapy, having serum total 25-hydroxyvitamin D less than 30 ng/mL.

99. The method of claim 98, wherein the patient has serum total 25-hydroxyvitamin D at least 10 ng/mL at initiation of therapy.

100. The method of claim 83, wherein the amount of 25-hydroxyvitamin D administered is effective to achieve a serum total 25-hydroxyvitamin D level in mean in the population, up to about 300 ng/mL.

101. The method of claim 82, wherein the patient has CKD Stage 3 to 5, or Stage 3 to 4, or Stage 5.

102. The method of claim 82, wherein the patient is also being treated by hemodialysis.

103. The method of claim 82, wherein the administration of 25-hydroxyvitamin D includes, consists essentially of, or consists of administration of 25-hydroxyvitamin D3.

104. The method of claim 82, wherein the administration of 25-hydroxyvitamin D comprises modified release administration.

105. The method of claim 82, wherein the administration of 25-hydroxyvitamin D comprises sustained release administration.

106. The method of claim 82, wherein the administration of 25-hydroxyvitamin D comprises oral administration.

107. The method of claim 82, wherein the administration of 25-hydroxyvitamin D comprises administering 25-hydroxyvitamin D intravenously over an extended period of time, optionally over a period of at least 1 hour, optionally up to 5 hours.

108. The method of claim 82, wherein the administration of 25-hydroxyvitamin D comprises avoiding substantial induction of CYP24A1, optionally characterized by a VMR of 5 or less, or 4.8 or less.

109. The method of claim 82, wherein the 25-hydroxyvitamin D is administered at a frequency of daily or less frequently.

110. The method of claim 82, wherein the 25-hydroxyvitamin D is administered daily.

111. The method of claim 82, wherein the 25-hydroxyvitamin D is administered 3 times per week.

112. The method of claim 82, wherein the 25-hydroxyvitamin D is administered in a unit dose form comprising 30 μg to 600 μg of 25-hydroxyvitamin D.

113. The method of claim 82, comprising administering 25-hydroxyvitamin D in a range of about 300 μg to about 900 μg per week.

114. The method of claim 82, comprising administering a weekly dose of 25-hydroxyvitamin D divided into two or three doses per week, optionally three times per week at dialysis treatment.

115. The method of claim 82, comprising reducing the blood level of a bone resorption marker in the patient.

116. The method of claim 115, wherein the bone resorption marker is one or more marker selected from serum total alkaline phosphatase, BSAP, CTX-1, P1NP, and FGF-23.

117. The method of claim 115, wherein the reduction is to within the normal reference range for the marker.

118. The method of claim 115, wherein the reduction is by at least about 10%, or at least about 20%, or at least about 30%.
